(19) United States
(12) Patent Application Publication
(10) Pub. No.: US 2024/0024640 A1
(43) Pub. Date: Jan. 25, 2024

(54) DYNAMICALLY RIGIDIZING GUIDERAIL AND METHODS OF USE

(71) Applicant: NEPTUNE MEDICAL INC., Burlingame, CA (US)

(72) Inventors: Garrett J. GOMES, Pleasant Hill, CA (US); Alexander Q. TILSON, Burlingame, CA (US); Charles Steele LOVE, Santa Barbara, CA (US); Deanna K. HENDRICKSON, Santa Clara, CA (US); Alberto R. CANTU, San Francisco, CA (US)

(21) Appl. No.: 18/044,027

(22) PCT Filed: Sep. 3, 2021
(86) PCT No.: PCT/US2021/049165

§ 371 Date: Mar. 3, 2023

Related U.S. Application Data
(60) Provisional application No. 63/074,422, filed on Sep. 3, 2020.

(51) Int. Cl. A61M 25/09; A61B 17/22; A61M 5/00; A61F 2/24

(52) U.S. Cl. CPC A61M 25/09041; A61B 17/22; A61M 5/007; A61F 2/2427; A61B 2017/22038; A61B 2017/22079; A61M 2210/125; A61M 2025/09116; A61M 2025/0915; A61B 10/02

(57) ABSTRACT

A rigidizing guiderail includes a rigidizing elongate tube having a tubular inner layer, a stiffening layer positioned radially outwards of the tubular inner layer, an outer layer over the tubular inner layer and the stiffening layer, and a vacuum or pressure inlet between the tubular inner layer and tire outer layer and configured to attach to a source of vacuum or pressure. The inner diameter of the tubular inner layer forms a guide wire lumen. The rigidizing elongate tube is configured to have a rigid configuration when vacuum or pressure is applied through the inlet and a flexible configuration when vacuum or pressure is not applied through the inlet.

### CROSS REFERENCE TO RELATED APPLICATIONS

This application claims priority to U.S. Provisional Patent Application No. 63/074,422, filed on Sep. 3, 2020, titled “DYNAMICALLY RIGIDIZING GUIDER AIL AND METHODS OF USE,” the entirety of which is incorporated by reference herein.

This application may also be related to International Application No. PCT/US2019/042650, filed on Jul. 19, 2019, titled “DYNAMICALLY RIGIDIZING COMPOSITE MEDICAL STRUCTURES,” and International Application No. PCT/US2020/013937, filed on Jan. 16, 2020, titled “DYNAMICALLY RIGIDIZING COMPOSITE MEDICAL STRUCTURES,” the entireties of which are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Most transcatheter procedures rely on the use of a pre-placed guidewire to direct and navigate the catheter to the desired anatomy. For example, prior to deploying a stent in the coronary artery, a guidewire is advanced through the lesion to serve as a guide for the catheter carrying the stent.

Guidewires come in a range of flexibilities that are utilized for different procedures amongst different anatomies. Often, a flexible guidewire is used to gain initial access, and then replaced with a stiffer guidewire to provide greater support. However, guidewires can be overcome by the force of the catheter it is guiding and can straighten as the catheter is passed over it, particularly as larger and less flexible catheters place more force on the guidewire, and particularly when navigating through tortuous anatomy. Guidewire displacement, including losing guidewire position, can affect the ability to place the catheter in the correct position and can significantly extend procedural difficulty, risk, complications, and duration.

Moreover, current guidewires and guide catheters are often not effective for large bore catheters (e.g., large bore catheters for pulmonary embolisms) because the large bore catheter, due to its increased stiffness and large bore size, cannot easily track over the guidewire through tortuous anatomy (e.g., through the right atrium, the tricuspid valve, the right ventricle, the pulmonary valve, and through the pulmonary bifurcations). As a result, other more difficult or complicated treatments are often required. For example, pulmonary embolism treatment often is treated with either thrombolytics (which are very expensive, have potentially deadly complications, require an ICU stay, and are not particularly effective for larger or older clots), or surgery (which is very traumatic and expensive, including a sternotomy, bypass, an extended stay in the hospital, and prolonged recovery).

Finally, current guidewires tend to require a tradeoff between having high flexibility (which is gentler on the anatomy, but offers less support) and high stiffness (which offers greater support, but can damage the anatomy).

Accordingly, a device that addresses some or all of these issues is desired so as to enable enhanced procedural access, navigation, and/or subsequent treatment.

### SUMMARY OF THE DISCLOSURE

In general, in one embodiment, a rigidizing guiderail includes a rigidizing elongate tube having a tubular inner layer, a stiffening layer positioned radially outwards of the tubular inner layer, an outer layer over the tubular inner layer and the stiffening layer, and a vacuum or pressure inlet between the tubular inner layer and the outer layer and configured to attach to a source of vacuum or pressure. The inner diameter of the tubular inner layer forms a guidewire lumen. The rigidizing elongate tube is configured to have a rigid configuration when vacuum or pressure is applied through the inlet and a flexible configuration when vacuum or pressure is not applied through the inlet.

This and other embodiments can include one or more of the following features. The rigidizing guiderail can further include a tapered distal tip connected to the rigidizing elongate tube. The tapered distal tip can taper at an angle of 5-45 degrees relative to a longitudinal axis of the rigidizing elongate tube. A ratio of an inner diameter of the tubular inner layer to an outer diameter of the rigidizing elongate tube can be less than 50%. A ratio of a double wall thickness of the rigidizing elongate tube to an outer diameter of the rigidizing elongate tube can be greater than 60%. The stiffening layer can be a braid layer. The rigidizing guiderail can further include a bladder layer configured to push the stiffening layer against the outer layer when pressure is supplied to the inlet. A distal portion of the rigidizing guiderail can be configured to be steerable. The rigidizing guiderail can further include a distal balloon attached thereto. A system can include the rigidizing guiderail and a guidewire configured to extend through the guidewire lumen. The rigidizing guiderail in the rigid configuration can have a higher stiffness than the guidewire. The rigidizing guiderail in the flexible configuration can have a lower stiffness than the guidewire.

In general, in one embodiment, a method of performing a medical procedure includes inserting a guidewire into a body lumen to a desired location, inserting a rigidizing guiderail over the guidewire while the rigidizing guiderail is in a flexible configuration, activating pressure or vacuum to transition the rigidizing guiderail to a rigid configuration when the rigidizing guiderail is proximate to the desired location, passing a catheter over the rigidizing guiderail while the rigidizing guiderail is in the rigid configuration, and performing a medical procedure using the catheter.

This and other embodiments can include one or more of the following features. The rigidizing guiderail in the rigid configuration can have a higher stiffness than the guidewire. The rigidizing guiderail in the flexible configuration can have a lower stiffness than the guidewire. The method can further include steering the rigidizing guiderail with a steering element while the rigidizing guiderail is positioned over the guidewire. The method can further include rigidizing the catheter by activating pressure or vacuum. The method can further include inserting a third device through the catheter to perform the medical procedure. The third device can be an aspiration catheter. The method can further include releasing the pressure or vacuum to transition the rigidizing guiderail back to the flexible configuration. The method can further include removing the rigidizing guiderail from the catheter prior to performing the medical procedure. The rigidizing guiderail can have a radial gap of 0.0005″ to 0.06″ around the guidewire. The body lumen can be a portion of the pulmonary vasculature. Performing a medical procedure can include treating for a pulmonary embolism. Performing a medical procedure can include treating chronic thromboembolic pulmonary hypertension (CTEPH). The method can further include puffing contrast through the rigidizing guiderail to identify a clot. Performing a medical procedure can include performing transcatheter aortic valve replacement. The body lumen can include an aortic bifurcation. Performing a medical procedure can include an electrophysiology procedure. The body lumen can be a portion of the neurovasculature.

In general, in one embodiment, a method of treating a pulmonary embolism includes inserting a guidewire into a body lumen to the pulmonary vasculature proximate to a pulmonary embolism, inserting a rigidizing guiderail over the guidewire while the rigidizing guiderail is in a flexible configuration, rigidizing the rigidizing guiderail to a rigid configuration when the rigidizing guiderail is proximate to the pulmonary embolism, passing a catheter over the rigidizing guiderail while the rigidizing guiderail is in the rigid configuration, and removing at least a portion of the pulmonary embolism through the catheter.

This and other embodiments can include one or more of the following features. The method can further include steering the rigidizing guiderail with a steering element while the guiderail is positioned over the guidewire. Rigidizing the guiderail can include rigidizing by activating pressure or vacuum. The method can further include rigidizing the catheter by activating pressure or vacuum. The step of removing at least a portion of the pulmonary embolism can be performed with a third device inserted through the catheter. The third device can be an aspiration catheter. The method can further include transitioning the rigidizing guiderail back to the flexible configuration. The method can further include removing the rigidizing guiderail from the catheter prior to the step of removing at least a portion of the pulmonary embolism through the catheter. The method can further include puffing contrast through the rigidizing guiderail to identify the pulmonary embolism. The method can further include, after the introducing step, inflating a balloon on a distal end of the rigidizing guiderail such that blood flow propels the balloon and rigidizing guiderail through the pulmonary vasculature.

In general, in one embodiment, a method of performing a medical procedure includes inserting a guidewire into a body lumen to a desired location, inserting a rigidizing guiderail over the guidewire while the rigidizing guiderail is in a flexible configuration, activating pressure or vacuum to transition the rigidizing guiderail to a rigid configuration when the rigidizing guiderail is proximate to the desired location, removing the guidewire from the central lumen, and performing a medical procedure through the central lumen.

This and other embodiments can include one or more of the following features. Performing a medical procedure can include inserting a biopsy tool through the central lumen to gather a tissue sample for biopsy. Performing a medical procedure can include aspirating a clot through the central lumen. The rigidizing guiderail in the rigid configuration can have a higher stiffness than the guidewire. The rigidizing guiderail in the flexible configuration can have a lower stiffness than the guidewire. The method can further include steering the rigidizing guiderail with a steering element while the guiderail is positioned over the guidewire. The method can further include releasing the pressure or vacuum to transition the rigidizing guiderail back to the flexible configuration. The rigidizing guiderail can have a radial gap of 0.0005″ to 0.006″ around the guidewire. The body lumen can be a portion of the pulmonary vasculature. The body lumen can be a portion of the neurovasculature. The body lumen can be a myocardium. The body lumen can be a coronary ostium.

In general, in one embodiment, a rigidizing guidewire includes a rigidizing elongate member with no axial through-lumen. The rigidizing elongate member has an outer layer, a stiffening layer within the outer layer, and a vacuum or pressure gap within the outer layer and configured to attach to a source of vacuum or pressure. The rigidizing elongate member is configured to have a rigid configuration when vacuum or pressure is applied through the inlet and a flexible configuration when vacuum or pressure is not applied through the inlet.

This and other embodiments can include one or more of the following features. The rigidizing guidewire can further include a tapered distal tip connected to the rigidizing elongate member. The tapered distal tip can taper at an angle of 5-45 degrees relative to a longitudinal axis of the rigidizing elongate member. The stiffening layer can be a braid layer. The rigidizing guidewire can further include a bladder layer configured to push the stiffening layer against the outer layer when pressure is supplied to the gap. A distal portion of the rigidizing guidewire can be configured to be steerable. The rigidizing guidewire can further include a distal balloon attached thereto.

In general, in one embodiment, a method of performing a medical procedure includes inserting a rigidizing guidewire in a vessel to a target location while the rigidizing guidewire is in a flexible configuration, activating pressure or vacuum to transition the rigidizing guidewire to a rigid configuration when the rigidizing guidewire is proximate to the desired location, passing a catheter over the rigidizing guidewire while the rigidizing guidewire is in the rigid configuration, transitioning the rigidizing guidewire to a flexible configuration, removing the rigidizing guidewire from the vessel, and performing a medical procedure using the catheter. The rigidizing guidewire has no axial through-lumen.

This and other embodiments can include one or more of the following features. The method can further include steering the rigidizing guidewire with a steering element. The method can further include rigidizing the catheter by activating pressure or vacuum.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a rigidizing device.

FIGS. 2A-2B show exemplary rigidized shapes of a rigidizing device.

FIGS. 3A-3D show an exemplary vacuum rigidizing device.

FIGS. 4A-4B show an exemplary pressure rigidizing device.

FIG. 5 shows a nested rigidizing system.

FIG. 6 shows a nested rigidizing system with a cover between the inner and outer rigidizing devices.

FIGS. 7A-7B show a nested rigidizing system where the outer rigidizing device includes steering and imaging.

FIGS. 8A-8H show exemplary use of a nested rigidizing system.

FIGS. 9A-9B show an exemplary rigidizing guiderail.

FIG. 10 shows the rigidizing guiderail with a proximal hub attached thereto.

FIGS. 11A-11E show exemplary use of a rigidizing guiderail in the pulmonary vasculature.

FIGS. 12A-12B show an exemplary obturator.

FIG. 13 shows exemplary placement of a rigidizing guiderail in the pulmonary vasculature.

FIGS. 14A-14D show another exemplary obturator.

FIG. 15 shows the distal end of an exemplary steerable rigidizing guiderail.

FIG. 16 shows the distal end of an exemplary rigidizing guidewire.

FIG. 17 shows a rigidizing guiderail with contrast dye puffed therethrough.

FIG. 18 shows the distal end of a rigidizing guiderail with a biopsy tool extending therethrough.

FIG. 19 shows the distal end of a rigidizing guiderail with a non-braid stiffening layer.

FIG. 20 shows the distal end of a rigidizing guiderail with a balloon thereon.

FIG. 21 shows the distal end of a rigidizing guiderail configured to rigidize via a change in temperature.

FIG. 22 shows the distal end of a rigidizing device that is naturally stiff and transitioned to the flexible configuration via the application of pressure or vacuum.

FIGS. 23A-23F show a rigidizing guiderail including two steering sections.

FIG. 24 shows an exemplary use of a rigidizing guiderail for transcaval transcatheter aortic valve replacement (TAVR).

FIGS. 25A-25H show an exemplary method of using a rigidizing guiderail for treating chronic thromboembolic pulmonary hypertension with a rigidizing guiderail.

FIGS. 26A-26C show an exemplary method of using a rigidizing guiderail for transfemoral TAVR.

FIGS. 27A-27B show an exemplary method of using a rigidizing guiderail to insert an arterial stent up and over the aortic bifurcation.

### DETAILED DESCRIPTION

In general, described herein are rigidizing devices that are configured to aid in transporting or guiding a medical instrument through a curved, looped, or unsupported or poorly supported portion of the body (e.g., a vessel). The rigidizing devices can be long, thin, and hollow and can transition quickly from a flexible configuration (i.e., one that is relaxed, limp, or floppy) to a rigid configuration (i.e., one that is stiff and/or holds the shape it is in when it is rigidized). A plurality of layers (e.g., braided layers, bladder layers and/or an outer layer) can together form the wall of the rigidizing devices. The rigidizing devices can transition from the flexible configuration to the rigid configuration, for example, by applying a vacuum or pressure to the wall of the rigidizing device or within the wall of the rigidizing device. With the vacuum or pressure removed, the layers can easily shear or move relative to each other. With the vacuum or pressure applied, the layers can transition to a condition in which they exhibit substantially enhanced ability to resist shear, movement, bending, and buckling, thereby providing system rigidization. In some embodiments, multiple rigidizing devices can be used together as a nested system (i.e., with one rigidizing device positioned radially within another rigidizing system) to enhance the ability to reach tortuous locations in the body.

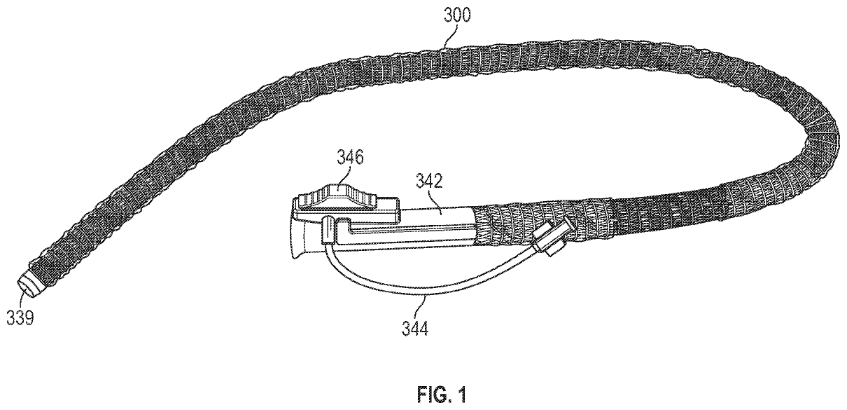

An exemplary rigidizing device system is shown in FIG. 1. The system includes a rigidizing device 300 having a wall with a plurality of layers including a braid layer, an outer layer (part of which is cut away to show the braid thereunder), and an inner layer. The system further includes a handle 342 having a vacuum or pressure inlet 344 to supply vacuum or pressure to the rigidizing device 300. An actuation element 346 can be used to turn the vacuum or pressure on and off to thereby transition the rigidizing device 300 between flexible and rigid configurations. The distal tip 339 of the rigidizing device 300 can be smooth, flexible, and atraumatic to facilitate distal movement of the rigidizing device 300 through the body. Further, the tip 339 can taper from the distal end to the proximal end to further facilitate distal movement of the rigidizing device 300 through the body.

In use, vacuum or pressure can be supplied between the walls of the rigidizing devices described herein, causing the braided layer and neighboring layer(s) to constrict and/or separate to transition between flexible and rigid configurations. The rigidizing devices described herein can thus advantageously transition from very flexible to very stiff upon activation by the user. When a vacuum or pressure is applied, the braids or strands can radially constrict or expand to become mechanically fixed or locked in place relative to one another. As a result, the rigidizing device can go from a flexible configuration to a rigid configuration when vacuum or pressure is applied (thereby fixing the rigidizing device in the shape that the rigidizing device was in just prior to application of the vacuum or pressure).

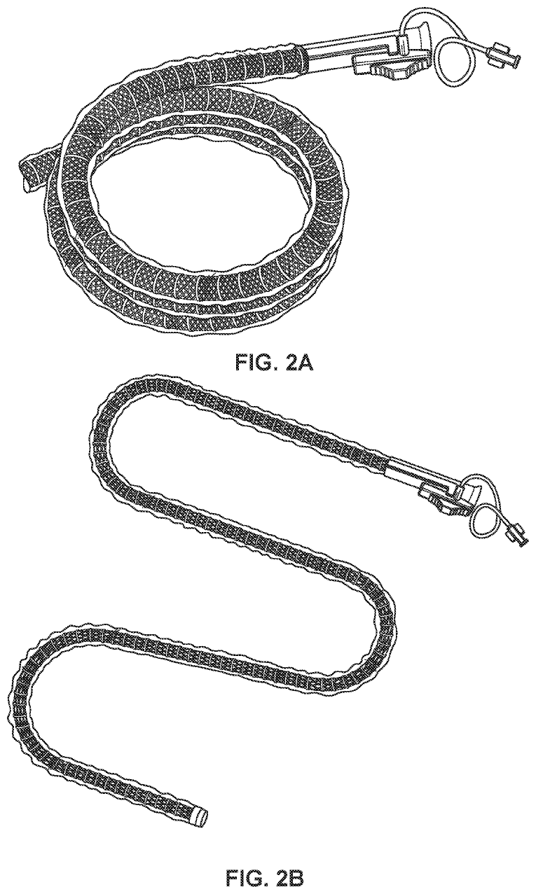

Exemplary rigidizing devices in the rigidized configuration are shown in FIGS. 2A and 2B. As the rigidizing device is rigidized, it does so in the shape it was in before vacuum or pressure was applied, i.e., it does not straighten, bend, or otherwise substantially modify its shape (e.g., it may stiffen in a looped configuration as shown in FIG. 2A or in a serpentine shape as shown in FIG. 2B). Upon release of the vacuum or pressure, braids or strands can unlock relative to one another and again move so as to allow bending of the rigidizing device. Again, as the rigidizing device is made more flexible through the release of vacuum or pressure, it does so in the shape it was in before the vacuum or pressure was released, i.e., it does not straighten, bend, or otherwise substantially modify its shape. Thus, the rigidizing devices described herein can transition from a flexible, less-stiff configuration to a rigid configuration of higher stiffness by restricting the motion between the strands of braid (e.g., by applying vacuum or pressure).

The rigidizing devices described herein can toggle between the rigid and flexible configurations quickly, and in some embodiments with an indefinite number of transition cycles. As interventional medical devices are made longer and inserted deeper into the human body, and as they are expected to do more exacting therapeutic procedures, there is an increased need for precision and control. Selectively rigidizing devices (e.g., overtubes, catheters, or guiderails) as described herein can advantageously provide both the benefits of flexibility (when needed) and the benefits of stiffness (when needed). Further, the rigidizing devices described herein can be used, for example, with classic endoscopes, colonoscopes, robotic systems, and/or navigation systems, catheters, or trocars, such as those described in International Patent Application No. PCT/US2016/050290, filed Sep. 2, 2016, titled “DEVICE FOR ENDOSCOPIC ADVANCEMENT THROUGH THE SMALL INTESTINE,” the entirety of which is incorporated by referenced herein.

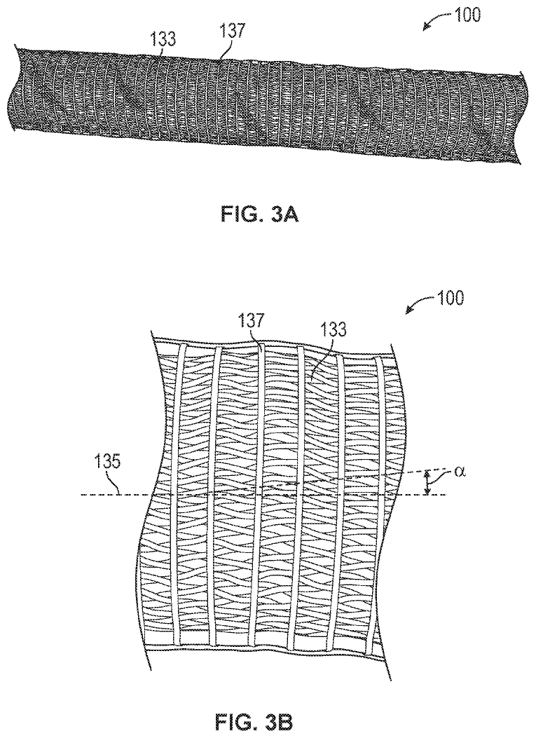

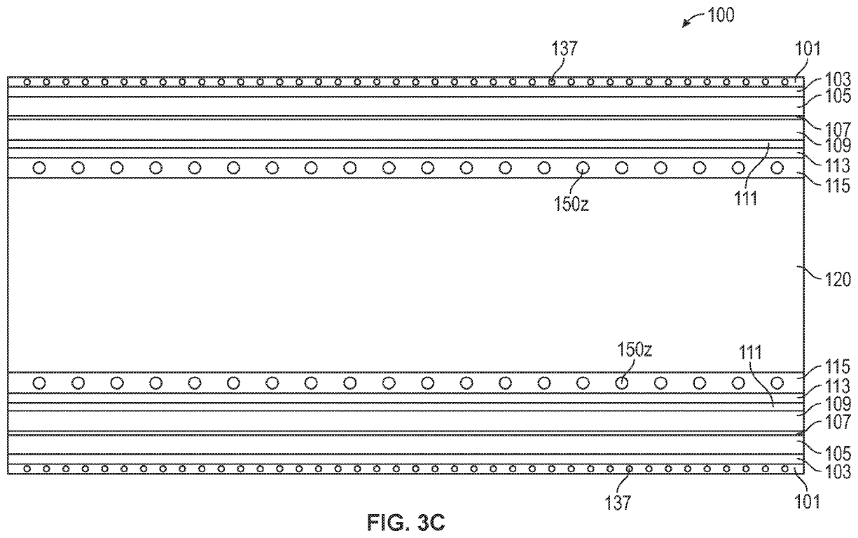

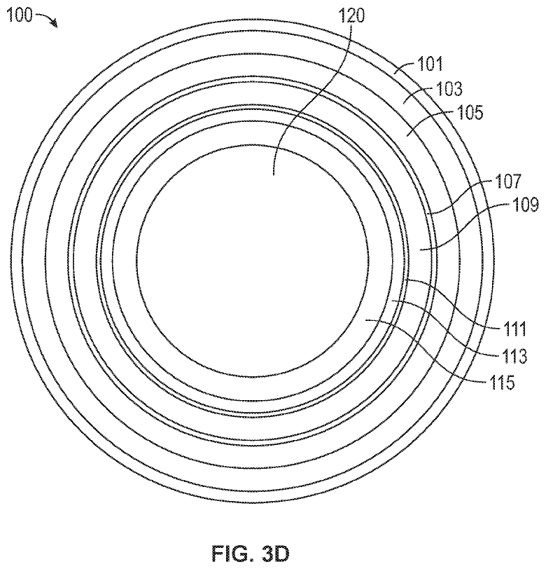

Referring to FIGS. 3A-3D, in one embodiment, a tubular rigidizing device 100 can include a wall having a plurality of layers positioned around the lumen 120 (e.g., for placement of an instrument or endoscope therethrough). A vacuum can be supplied between the layers to rigidize the rigidizing device 100.

The innermost layer 115 can be configured to provide an inner surface against which the remaining layers can be consolidated, for example, when a vacuum is applied within the walls of the rigidizing device 100. The structure can be configured to minimize bend force/maximize flexibility in the non-vacuum condition. In some embodiments, the innermost layer 115 can include a reinforcement element 150z or coil within a matrix, as described above.

The layer 113 over (i.e., radially outwards of) the innermost layer 115 can be a slip layer.

The layer 111 can be a radial gap (i.e., a space). The gap layer 111 can provide space for the braided layer(s) thereover to move within (when no vacuum is applied) as well as space within which the braided or woven layers can move radially inward (upon application of vacuum).

The layer 109 can be a first braid layer including braided strands 133 similar to as described elsewhere herein. The braid layer can be, for example, 0.001″ to 0.040″ thick. For example, a braid layer can be 0.001″, 0.003″, 0.005″, 0.010″, 0.015″, 0.020″, 0.025″ or 0.030″ thick.

In some embodiments, as shown in FIG. 3B, the braid can have tensile or hoop fibers 137. Hoop fibers 137 can be spiraled and/or woven into a braid layer. Further, the hoop fibers 137 can be positioned at 2-50, e.g., 20-40 hoops per inch. The hoop fibers 137 can advantageously deliver high compression stiffness (to resist buckling or bowing out) in the radial direction, but can remain compliant in the direction of the longitudinal axis 135 of the rigidizing device 100. That is, if compression is applied to the rigidizing device 100, the braid layer 109 will try to expand in diameter as it compresses. The hoop fibers 137 can resist this diametrical expansion and thus resist compression. Accordingly, the hoop fiber 137 can provide a system that is flexible in bending but still resists both tension and compression.

The layer 107 can be another radial gap layer similar to layer 111.

In some embodiments, the rigidizing devices described herein can have more than one braid layer. For example, the rigidizing devices can include two, three, or four braid layers. Referring to FIG. 3C, the layer 105 can be a second braid layer 105. The second braid layer 105 can have any of the characteristics described with respect to the first braid layer 109. In some embodiments, the braid of second braid layer 105 can be identical to the braid of first braid layer 109. In other embodiments, the braid of second braid layer 105 can be different than the braid of the first braid layer 109. For example, the braid of the second braid layer 105 can include fewer strands and have a larger braid angle α than the braid of the first braid layer 109. Having fewer strands can help increase the flexibility of the rigidizing device 100 (relative to having a second strand with equivalent or greater number of strands), and a larger braid angle α can help constrict the diameter of the of the first braid layer 109 (for instance, if the first braid layer is compressed) while increasing/maintaining the flexibility of the rigidizing device 100. As another example, the braid of the second braid layer 105 can include more strands and have a larger braid angle α than the braid of the first braid layer 109. Having more strands can result in a relatively tough and smooth layer while having a larger braid angle α can help constrict the diameter of the first braid layer 109.

The layer 103 can be another radial gap layer similar to layer 111. The gap layer 103 can have a thickness of 0.0002-0.04″, such as approximately 0.03″. A thickness within this range can ensure that the strands 133 of the braid layer(s) can easily slip and/or bulge relative to one another to ensure flexibility during bending of the rigidizing device 100.

The outermost layer 101 can be configured to move radially inward when a vacuum is applied to pull down against the braid layers 105, 109 and conform onto the surface(s) thereof. The outermost layer 101 can be soft and atraumatic and can be sealed at both ends to create a vacuum-tight chamber with layer 115. The outermost layer 101 can be elastomeric, e.g., made of urethane. The hardness of the outermost layer 101 can be, for example, 30 A to 80 A. Further, the outermost layer 101 can be have a thickness of 0.0001-0.01″, such as approximately 0.001″, 0.002, 0.003″ or 0.004″. Alternatively, the outermost layer can be plastic, including, for example, LDPE, nylon, or PEEK.

In some embodiments, the outermost layer 101 can, for example, have tensile or hoop fibers 137 extending therethrough. The hoop fibers 137 can be made, for example, of aramids (e.g., Technora, nylon, Kevlar), Vectran, Dyneema, carbon fiber, metal, fiber glass or plastic. Further, the hoop fibers 137 can be positioned at 2-50. e.g., 20-40 hoops per inch. In some embodiments, the hoop fibers 137 can be laminated within an elastomeric sheath. The hoop fibers can advantageously deliver higher stiffness in one direction compared to another (e.g., can be very stiff in the hoop direction, but very compliant in the direction of the longitudinal axis of the rigidizing device). Additionally, the hoop fibers can advantageously provide low hoop stiffness until the fibers are placed under a tensile load, at which point the hoop fibers can suddenly exhibit high hoop stiffness.

In some embodiments, the outermost layer 101 can include a lubrication, coating and/or powder (e.g., talcum powder) on the outer surface thereof to improve sliding of the rigidizing device through the anatomy. The coating can be hydrophilic (e.g., a Hydromer® coating or a Surmodics® coating) or hydrophobic (e.g., a fluoropolymer). The coating can be applied, for example, by dipping, painting, or spraying the coating thereon.

The innermost layer 115 can similarly include a lubrication, coating (e.g., hydrophilic or hydrophobic coating), and/or powder (e.g., talcum powder) on the inner surface thereof configured to allow the bordering layers to more easily shear relative to each other, particularly when no vacuum is applied to the rigidizing device 100, to maximize flexibility.

In some embodiments, the outermost layer 101 can be loose over the radially inward layers. For instance, the inside diameter of layer 101 (assuming it constitutes a tube) may have a diametrical gap of 0″-0.200″ with the next layer radially inwards (e.g., with a braid layer). This may give the vacuum rigidized system more flexibility when not under vacuum while still preserving a high rigidization multiple. In other embodiments, the outermost layer 101 may be stretched some over the next layer radially inwards (e.g., the braid layer). For instance, the zero-strain diameter of a tube constituting layer 101 may be from 0-0.200″ smaller in diameter than the next layer radially inwards and then stretched thereover. When not under vacuum, this system may have less flexibility than one wherein the outer layer 101 is looser. However, it may also have a smoother outer appearance and be less likely to tear during use.

In some embodiments, the outermost layer 101 can be loose over the radially inward layers. A small positive pressure may be applied underneath the layer 101 in order to gently expand layer 101 and allow the rigidizing device to bend more freely in the flexible configuration. In this embodiment, the outermost layer 101 can be elastomeric and can maintain a compressive force over the braid, thereby imparting stiffness. Once positive pressure is supplied (enough to nominally expand the sheath off of the braid, for example, 2 psi), the outermost layer 101 is no longer is a contributor to stiffness, which can enhance baseline flexibility. Once rigidization is desired, positive pressure can be replaced by negative pressure (vacuum) to deliver stiffness.

A vacuum can be carried within rigidizing device 100 from minimal to full atmospheric vacuum (e.g., approximately 14.7 psi). In some embodiments, there can be a bleed valve, regulator, or pump control such that vacuum is bled down to any intermediate level to provide a variable stiffness capability. The vacuum pressure can advantageously be used to rigidize the rigidizing device structure by compressing the layer(s) of braided sleeve against neighboring layers. Braid is naturally flexible in bending (i.e. when bent normal to its longitudinal axis), and the lattice structure formed by the interlaced strands distort as the sleeve is bent in order for the braid to conform to the bent shape while resting on the inner layers. This results in lattice geometries where the corner angles of each lattice element change as the braided sleeve bends. When compressed between conformal materials, such as the layers described herein, the lattice elements become locked at their current angles and have enhanced capability to resist deformation upon application of vacuum, thereby rigidizing the entire structure in bending when vacuum is applied. Further, in some embodiments, the hoop fibers through or over the braid can carry tensile loads that help to prevent local buckling of the braid at high applied bending load.

The stiffness of the rigidizing device 100 can increase from 2-fold to over 30-fold, for instance 10-fold, 15-fold, or 20-fold, when transitioned from the flexible configuration to the rigid configuration. In one specific example, the stiffness of a rigidizing device similar to rigidizing device 100 was tested. The wall thickness of the test rigidizing device was 1.0 mm, the outer diameter was 17 mm, and a force was applied at the end of a 9.5 cm long cantilevered portion of the rigidizing device until the rigidizing device deflected 10 degrees. The force required to do so when in flexible mode was only 30 grams while the force required to do so in rigid (vacuum) mode was 350 grams.

In some embodiments of a vacuum rigidizing device 100, there can be only one braid layer. In other embodiments of a vacuum rigidizing device 100, there can be two, three, or more braid layers. In some embodiments, one or more of the radial gap layers or slip layers of rigidizing device 100 can be removed. In some embodiments, some or all of the slip layers of the rigidizing device 100 can be removed.

The braid layers described herein can act as a variable stiffness layer. The variable stiffness layer can include one or more variable stiffness elements or structures that, when activated (e.g., when vacuum is applied), the bending stiffness and/or shear resistance is increased, resulting in higher rigidity. Other variable stiffness elements can be used in addition to or in place of the braid layer. In some embodiments, engagers can be used as a variable stiffness element, as described in International Patent Application No. PCT/US2018/042946, filed Jul. 19, 2018, titled “DYNAMICALLY RIGIDIZING OVERTUBE,” the entirety of which is incorporated by reference herein. Alternatively or additionally, the variable stiffness element can include particles or granules, jamming layers, scales, rigidizing axial members, rigidizers, wedges, laser cut tubes, longitudinal members or substantially longitudinal members.

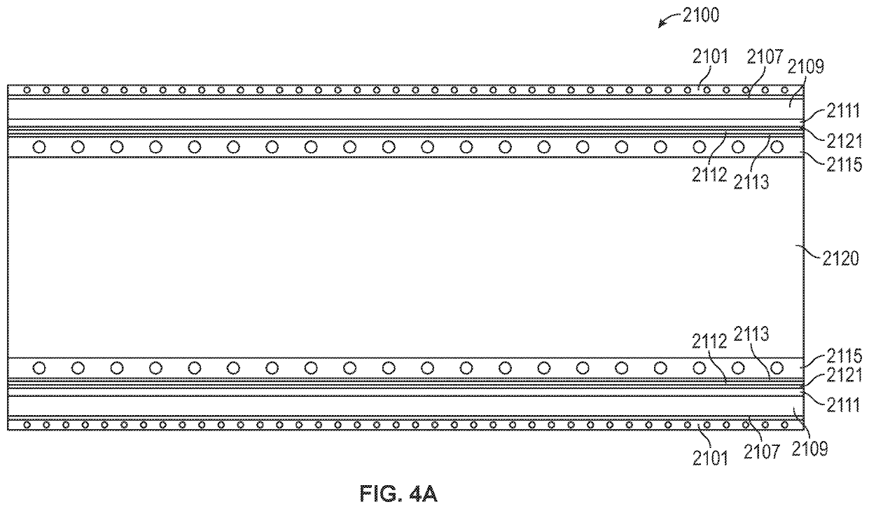

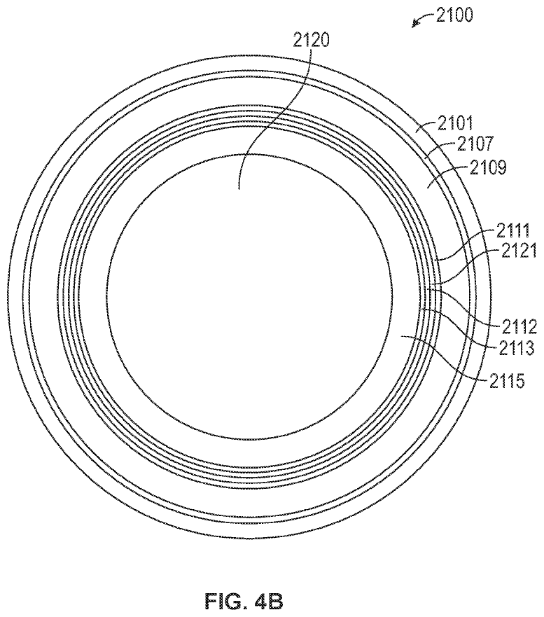

In some embodiments, the rigidizing devices described herein can rigidize through the application of pressure rather than vacuum. For example, referring to FIGS. 4A-4B, the rigidizing device 2100 can be similar to rigidizing device 100 except that it can be configured to hold pressure (e.g., of greater than 1 atm) therein for rigidization rather than vacuum. The rigidizing device 2100 can thus include a plurality of layers positioned around the lumen 2120 (e.g., for placement of an instrument or endoscope therethrough). The rigidizing device 2100 can include an innermost layer 2115 (similar to innermost layer 115), a slip layer 2113 (similar to slip layer 113), a pressure gap 2112, a bladder layer 2121, a gap layer 2111 (similar to gap layer 111), a braid layer 2109 (similar to braid layer 109) or other variable stiffness layer as described herein, a gap layer 2107 (similar to layer 107), and an outermost containment layer 2101.

The pressure gap 2112 can be a sealed chamber that provides a gap for the application of pressure to layers of rigidizing device 2100. The pressure can be supplied to the pressure gap 2112 using a fluid or gas inflation/pressure media. The inflation/pressure media can be water or saline or, for example, a lubricating fluid such as oil or glycerin. The lubricating fluid can, for example, help the layers of the rigidizing device 2100 flow over one another in the flexible configuration. The inflation/pressure media can be supplied to the gap 2112 during rigidization of the rigidizing device 2100 and can be partially or fully evacuated therefrom to transform the rigidizing device 2100 back to the flexible configuration. In some embodiments, the pressure gap 2112 of the rigidizing device 2100 can be connected to a pre-filled pressure source, such as a pre-filled syringe or a pre-filled insufflator, thereby reducing the physician's required set-up time.

The bladder layer 2121 can be made, for example, of a low durometer elastomer (e.g., of shore 20 A to 70 A, 80 A, or 90 A) or a thin plastic sheet (e.g., an extruded or blown thin plastic sheet). The bladder layer 2121 can be formed out of a thin sheet of plastic or rubber that has been sealed lengthwise to form a tube. The lengthwise seal can be, for instance, a butt or lap joint. For instance, a lap joint can be formed in a lengthwise fashion in a sheet of rubber by melting the rubber at the lap joint or by using an adhesive. In some embodiments, the bladder layer 2121 can be 0.0002″-0.020″ thick, such as approximately 0.005″ thick. The bladder layer 2121 can be soft, high-friction, stretchy, and/or able to wrinkle easily. In some embodiments, the bladder layer 2121 is a polyolefin or a PET (e.g., as thin as 0.0005″). The bladder 2121 can be formed, for example, by using methods used to form heat shrink tubing, such as extrusion of a base material and then wall thinning with heat, pressure and/or radiation. When pressure is supplied through the pressure gap 2112, the bladder layer 2121 can expand through the gap layer 2111 to push the braid layer 2109 against the outermost containment layer 2101 such that the relative motion of the braid strands is reduced.

The outermost containment layer 2101 can be a tube, such as an extruded tube. Alternatively, the outermost containment layer 2101 can be a tube in which a reinforcing member (for example, metal wire, including round or rectangular cross-sections) is encapsulated within an elastomeric matrix, similar to as described with respect to the innermost layer for other embodiments described herein. In some embodiments, the outermost containment layer 2101 can include a helical spring (e.g., made of circular or flat wire), and/or a tubular braid (such as one made from round or flat metal wire) and a thin elastomeric sheet that is not bonded to the other elements in the layer. The outermost containment layer 2101 can be a tubular structure with a continuous and smooth surface. This can facilitate an outer member that slides against it in close proximity and with locally high contact loads (e.g., a nested configuration as described further herein). Further, the outer layer 2101 can be configured to support compressive loads, such as pinching. Additionally, the outer layer 2101 (e.g., with a reinforcement element therein) can be configured to prevent the rigidizing device 2100 from changing diameter even when pressure is applied.

Because both the outer layer 2101 and the inner layer 2115 include reinforcement elements therein, the braid layer 2109 can be reasonably constrained from both shrinking diameter (under tensile loads) and growing in diameter (under compression loads).

By using pressure rather than vacuum to transition from the flexible state to the rigid state, the rigidity of the rigidizing device 2100 can be increased. For example, in some embodiments, the pressure supplied to the pressure gap 2112 can be between 1 and 40 atmospheres, such as between 2 and 40 atmospheres, such as between 4 and 20 atmospheres, such as between 5 and 10 atmospheres. In some embodiments, the pressure supplied is approximate 2 atm, approximately 4 atmospheres, approximately 5 atmospheres, approximately 10 atmospheres, approximately 20 atmospheres. In some embodiments, the rigidizing device 2100 can exhibit change in relative bending stiffness (as measured in a simple cantilevered configuration) from the flexible configuration to the rigid configuration of 2-100 times, such as 10-80 times, such as 20-50 times. For example, the rigidizing device 2100 can have a change in relative bending stiffness from the flexible configuration to the rigid configuration of approximately 10, 15, 20, or 25, 30, 40, 50, or over 100 times.

In some embodiments, the rigidizing devices described herein can be used in conjunction with other versions of the product. For example, an endoscope can include the rigidizing mechanisms described herein, and a rigidizing device can include the rigidizing mechanisms described herein. Used together, they can create a nested system that can advance, one after the other, allowing one of the elements to always remain stiffened, such that looping is reduced or eliminated (i.e., they can create a sequentially advancing nested system). As another example, a rigidizing guiderail or dilator can be used in conjunction with a large bore rigidizing catheter.

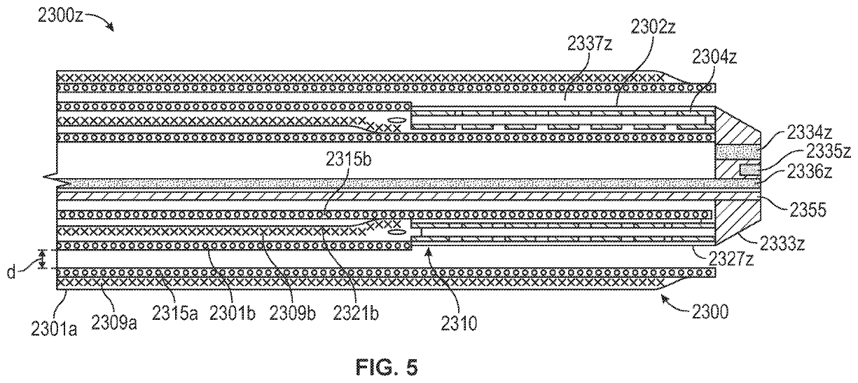

An exemplary nested system 2300z is shown in FIG. 5. The system 2300z can include an outer rigidizing device 2300 and an inner rigidizing device 2310 (here, configured as a rigidizing scope) that are axially movable with respect to one another either concentrically or non-concentrically. The outer rigidizing device 2300 and the inner rigidizing device 2310 can include any of the rigidizing features as described herein. For example, the outer rigidizing device 2300 can include an outermost layer 2301a, a braided layer 2309a, and an inner layer 2315a including a coil wound therethrough. The outer rigidizing device 2300 can be, for example, configured to receive vacuum between the outermost layer 2301a and the inner layer 2315a to provide rigidization. Similarly, the inner scope 2310 can include an outer layer 2301b (e.g., with a coil wound therethrough), a braid layer 2309b, a bladder layer 2321b, and an inner layer 2315b (e.g., with a coil wound therethrough). The inner scope 2310 can be, for example, configured to receive pressure between the bladder 2321b and the inner layer 2315b to provide rigidization. Further, an air/water channel 2336z and a working channel 2355 can extend through the inner rigidizing device 2310. Additionally, the inner rigidizing scope 2310 can include a distal section 2302z with a camera 2334z, lights 2335z, and steerable linkages 2304z. A cover 2327z can extend over the distal section 2302z. In another embodiment, the camera and/or lighting can be delivered in a separate assembly (e.g., the camera and lighting can be bundled together in a catheter and delivered down the working channel 2355 and/or an additional working channel to the distalmost end 2333z).

An interface 2337z can be positioned between the inner rigidizing device 2310 and the outer rigidizing device 2300. The interface 2337z can be a gap, for example, having a dimension d (see FIG. 5) of 0.001″-0.050″, such as 0.0020″, 0.005″, or 0.020″ thick. In some embodiments, the interface 2337z can be low friction and include, for example, powder, coatings, or laminations to reduce the friction. In some embodiments, there can be seals between the inner rigidizing device 2310 and outer rigidizing device 2300, and the intervening space can be pressurized, for example, with fluid or water, to create a hydrostatic hearing. In other embodiments, there can be seals between the inner rigidizing device 2310 and outer rigidizing device 2300, and the intervening space can be filled with small spheres to reduce friction.

The inner rigidizing device 2310 and outer rigidizing device 2300 can move relative to one another and alternately rigidize so as to transfer a bend or shape down the length of the nested system 2300z. For example, the inner device 2310 can be inserted into a lumen and bent or steered into the desired shape. Pressure can be applied to the inner rigidizing device 2310 to cause the braid elements to engage and lock the inner rigidizing device 2310 in the configuration. The rigidizing device (for instance, in a flexible state) 2300 can then be advanced over the rigid inner device 2310. When the outer rigidizing device 2300 reaches the tip of the inner device 2310, vacuum can be applied to the rigidizing device 2300 to cause the layers to engage and lock to fix the shape of the rigidizing device. The inner device 2310 can be transitioned to a flexible state, advanced, and the process repeated. Although the system 2300z is described as including a rigidizing device and an inner device configured as a scope, it should be understood that other configurations are possible. For example, the system might include two overtubes, two catheters, or a combination of overtube, catheter, and scope.

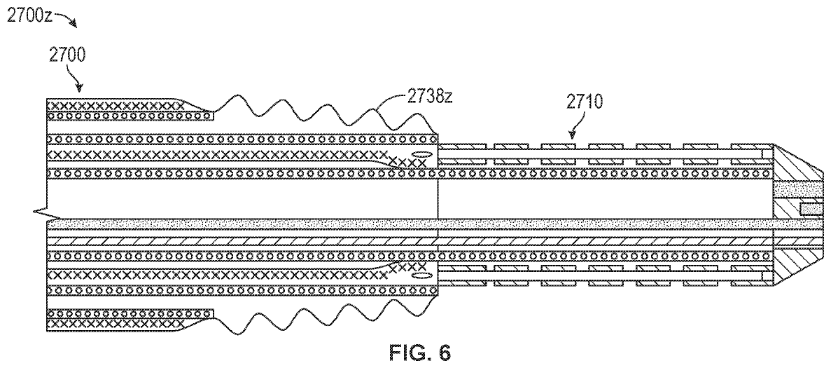

FIG. 6 shows another exemplary nested system 2700z. System 2700z is similar to system 2300z except that it includes a cover 2738z attached to both the inner and outer rigidizing device 2710, 2700. The cover 2738z may be, for example, low-durometer and thin-walled to allow elasticity and stretching. The cover 2738z may be a rubber, such as urethane, latex, or silicone. The cover 2738z may protect the interface/radial gap between the inner and outer devices 2710, 2700. The cover 2738z may prevent contamination from entering the space between the inner and outer tubes. The cover 2738z may further prevent tissue and other substances from becoming trapped in the space between the inner and outer tubes. The cover 2738z may stretch to allow the inner device 2710 and outer device 2700 to travel independently of one another within the elastic limits of the material. The cover 2738z may be bonded or attached to the rigidizing devices 2710, 2700 in such a way that the cover 2738z is always at a minimum slightly stretched. This embodiment may be wiped down externally for cleaning. In some embodiments, the cover 2738z can be configured as a “rolling” seal, such as disclosed in U.S. Pat. No. 6,447,491, the entire disclosure of which is incorporated by reference herein.

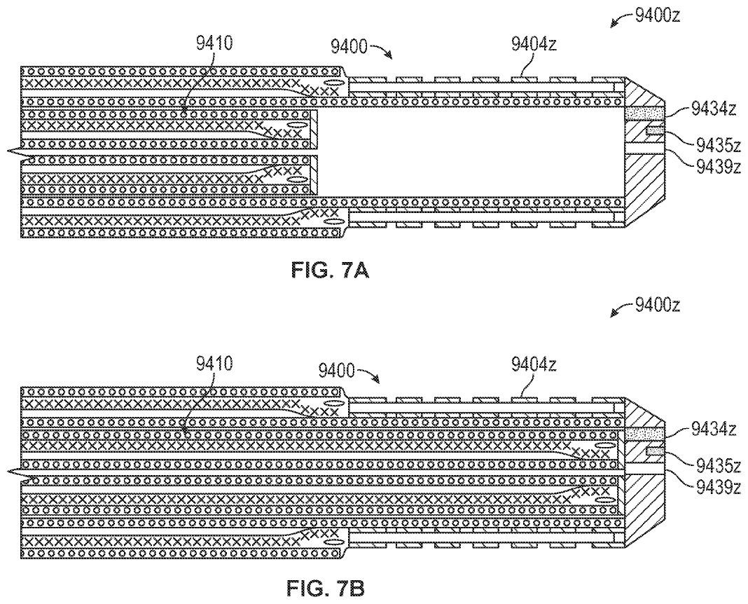

FIGS. 7A-7B show another exemplary nested system 9400z. In this system 9400z, the outer rigidizing device 9400 includes steering and imaging (e.g., similar to a scope) while the inner device includes only rigidization (though it could include additional steering elements as described elsewhere herein). Thus, outer device 9400 includes linkages or other steering means disclosed herein 9404z, camera 9434z, and lighting 9435z. The outer device 9400 can further include a central passageway 9439z for access to the inner device 9410 (e.g., lumens such as working channels therein). In some embodiments, bellows or a loop of tubing can connect the passageway 9439z to lumens of the inner device 9410. Similar to the other nested systems, at least one of the devices 9410, 9400 can be rigidized at a time while the other can conform to the rigidization and/or move through the anatomy. Here, the outer device 9400 can lead the inner device 9410 (the inner device 9410 is shown retracted relative to the outer device 9400 in FIG. 7A and extended substantially even with the outer device 9400 in FIG. 7B). Advantageously, system 9400z can provide a smooth exterior surface to avoid pinching the anatomy and/or entrance of fluid between the inner and outer devices 9410, 9400. Having the steering on the outer device 9400 can also provide additional leverage for steering the tip. Also, the outer device can facilitate better imaging capabilities due to the larger diameter of the outer device 9400 and its ability to accommodate a larger camera.

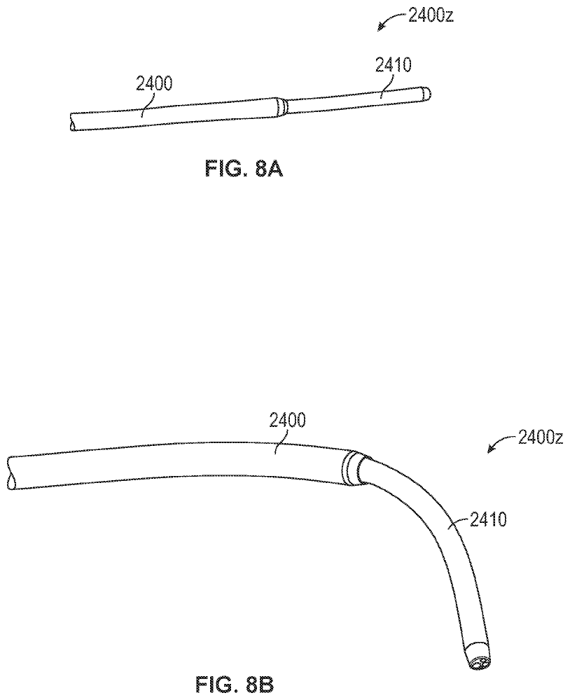

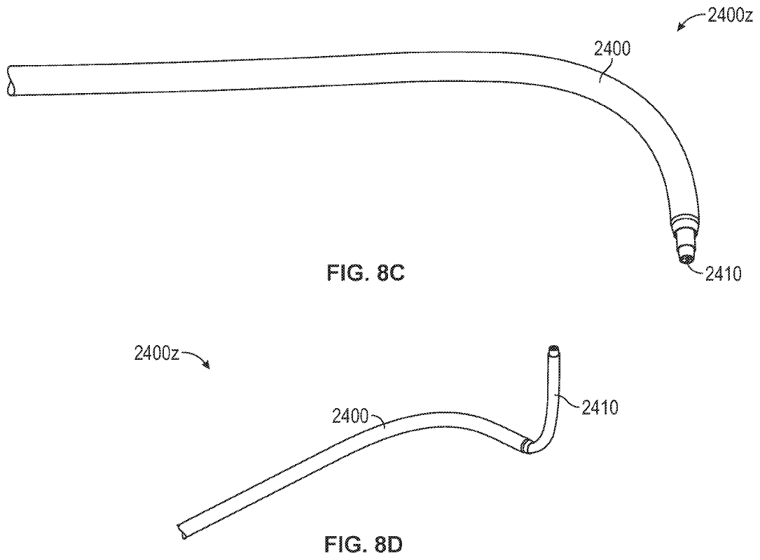

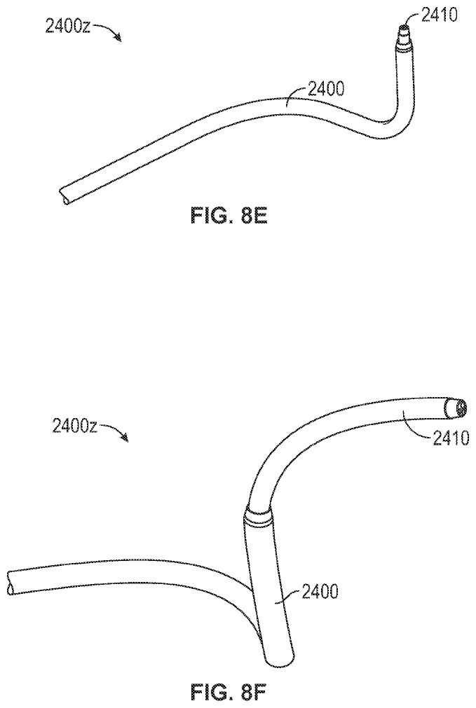

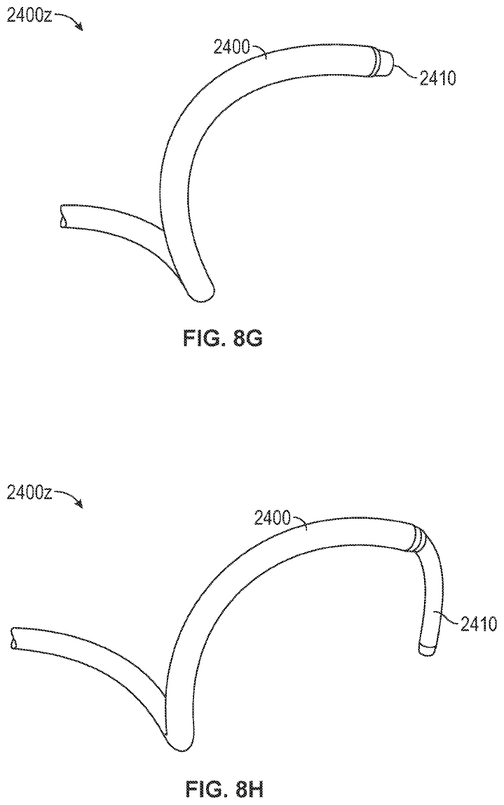

FIGS. 8A-8H show the exemplary use of a nested system 2400z as described herein. At FIG. 8A, the inner rigidizing device 2410 is positioned within the outer rigidizing device 2400 such that the distal end of the inner rigidizing device 2410 extends outside of the outer rigidizing device 2400. At FIG. 8B, the distal end of the inner rigidizing device 2410 is bent in the desired direction/orientation and then rigidized (e.g., using vacuum or pressure as described herein). At FIG. 8C, the outer rigidizing device 2400 (in the flexible configuration) is advanced over the rigidized inner rigidizing device 2410 (including over the bending distal section). Once the distal end of the outer rigidizing device 2400 is sufficiently advanced over the distal end of the inner rigidizing device 2410, then the outer rigidizing device 2400 can be rigidized (e.g., using vacuum or pressure as described herein). At FIG. 8D, the inner rigidizing device 2410 can then be transitioned to the flexible state (e.g., by removing the vacuum or pressure as described herein and by allowing the steering cables to go slack such that tip can move easily) and can be advanced and directed/oriented/steered as desired. Alternately, in FIG. 8D, the inner rigidizing device 2410 can be actively steered (either manually or via computational control) as it emerges such that is minimizes the load on the rigidized outer tube. Minimizing the load on the outer rigidizing device 2400 makes it easier for this tube to hold the rigidized shape. Once the inner rigidizing device 2410 is rigidized, the outer rigidizing device 2400 can be transitioned to the flexible state and advanced thereover (as shown in FIG. 8E). The process can then be repeated as shown in FIGS. 8F-H.

In some embodiments, at the completion of the sequence shown in FIGS. 8A-H, a third rigidizing device can be slid over the first two rigidizing devices (2400, 2410) and rigidized. Rigidizing devices 2400 and 2410 can then be withdrawn. Finally, a fourth rigidizing device can be inserted through the inner lumen of the third tube. This fourth rigidizing device may have a larger diameter and more features than rigidizing device 2410. For instance, it may have a larger working channel, more working channels, a better camera, or combinations thereof. This technique can allow two smaller tubes, which tend to be more flexible and maneuverable, to reach deep into the body while still ultimately deliver a larger tube for therapeutic purposes. Alternately, in the example above, the fourth rigidizing device can be a regular endoscope as is known in the art.

In some embodiments, at the completion of the sequence shown in FIGS. 8A-H, outer rigidizing device 2400 may be rigidized and then the inner rigidizing device 2410 may be removed. For example, the rigidizing device 2410 may be a “navigation” device comprising a camera, lighting and a distal steering section. The “navigation” device 2410 may be well sealed such that it is easy to clean between procedures. A second inner device may then be placed inside the rigidized outer device 2400 and advanced past the distal end of the outer device 2400. The second inner device may be a “therapeutic” tube comprising such elements as a camera, lights, water, suction and various tools. The “therapeutic” device may not have a steering section or the ability to rigidize, thereby giving additional room in the body of the therapeutic tube for the inclusion of other features, for example, tools for performing therapies. Once in place, the tools on the “therapeutic” tube may be used to perform a therapy in the body, such as, for example, a mucosal resection or dissection in the human GI tract.

In another embodiment, after or during the completion of the sequence shown in FIGS. 8A-H, a third device may be inserted inside inner tube 2410. The third device may be rigidizing and/or an endoscope.

In another embodiment, an outer rigidizing device can be stiffened with a biocompatible polymer that can harden to leave the rigidizing device in a permanent implantable configuration such as a cannula used on in a implantable heart assist pump.

Although the outer rigidizing device for the nested systems described herein is often referred to as rigidizing via vacuum and the inner scope rigidizing device as rigidizing via pressure, the opposite can be true (i.e., the outer rigidizing device can rigidize via pressure and the inner rigidizing device via vacuum) and/or both can have the same rigidizing source (pressure and/or vacuum).

Although the inner and outer elements of the nested systems are generally described as including integrated rigidizing elements, the rigidizing elements can be separate (e.g., so as to allow relative sliding between the imaging scope elements and the rigidizing elements).

The rigidizing devices of the nested systems described herein can be designed such that inner rigidizing device can't rotate substantially within outer rigidizing device when they are assembled. For instance, the outer surface of the inner rigidizing device can have longitudinal ridges and grooves that form a spline. The inner surface of the outer rigidizing device can have corresponding ridges and grooves that mate with the same features in the outer rigidizing device. In another embodiment, the rigidizing devices of the nested systems can be created of high torsional stiffness and can rotate relative to each other. For example, outer rigidizing device 2400 can be rigidized, and inner rigidizing device 2410 can be of high torsional stiffness, such that the inner rigidizing device 2410, while in the flexible configuration, can be effectively torqued within outer rigidizing device 2400. This torquing of the inner rigidizing device 2410 can provide optimized imaging (e.g., by keeping a camera on the inner rigidizing device 2410 parallel with the horizon) and/or optimized tool exit locations.

Either or both of the rigidizing devices of the nested systems described herein can be steerable, if both rigidizing devices are steerable, an algorithm can be implemented that steers whichever rigidizing device is flexible and moving longitudinally. The algorithm can steer the flexible rigidizing device to anticipate the shape of the rigidized device thus minimizing the tendency for the moving, flexible rigidizing device to straighten the rigid device.

If one rigidizing device of the nested systems described herein requires vacuum and the other rigidizing device requires pressure, user controls can be constructed in which moving one vs. the other (outer and inner) involves flipping a switch, with the switch toggling between a first condition in which, for example, one is pressurized for rigidity when the other is vented for flexibility and a second condition in which one is vented for flexibility and the other is vacuumed for stiffness. This, for example, could be a foot pedal or a hand switch.

In some embodiments, the alternate movement of the nested systems described herein can be controlled manually. In other embodiments, the alternate movement can be controlled automatically, via a computer and/or with a motorized motion control system, including as a motorized hand tool, or as a fully robotic system.

The nested systems described herein can advantageously be of similar stiffness. This can ensure that the total stiffnesses of the nested system is relatively continuous. The nested systems described herein can be small so as to fit in a variety of different anatomies. For example, for neurology applications, the outside diameter of the system can be between 0.05″ 0.15″, such as approximately 0.1″. For cardiology applications, the outside diameter of the system can be between 0.1″-0.3″, such as approximately 0.2″. For gastrointestinal applications, the outside diameter of the system can be between 0.3″-1.0″, such as 0.6″. Further, the nested systems described herein can maintain high stiffness even at a small profile. For example, the change in relative stiffness from the flexible configuration to the rigid configuration can be multiples of 10×, 20×, 30×, and even larger. Additionally, the nested systems described herein can advantageously move smoothly relative to one another.

The nested systems described herein can advantageously navigate an arbitrary path, or an open, complex, or tortuous space, and create a range of free-standing complex shapes. The nested systems can further advantageously provide shape propagation, allowing for shape memory to be imparted from one element to another, in some embodiments, periodically, both tubes can be placed in a partially or fully flexible state such that, for instance, the radii or curvature of the system increases, and the surrounding anatomy provides support to the system. The pressure or vacuum being used to rigidize the tubes can be reduced or stopped to place the tubes in a partially or fully flexible state. This momentary relaxation (for instance, for 1-10 seconds) may allow the system to find a shape that more closely matches the anatomy it is travelling through. For instance, in the colon, this relaxation may gently open tight turns in the anatomy.

In some embodiments, the stiffness capabilities of the inner or outer rigidizing devices may be designed such that tight turns formed by the inner rigidizing device at its tip, when copied by the outer rigidizing device, are gradually opened up (made to have a larger radius) as the shape propagates proximally down the outer tube. For instance, the outer rigidizing device may be designed to have a higher minimum radius of curvature when rigidized.

The nested systems are continuous (i.e., non-segmented) and therefor provide smooth and continuous movement through the body (e.g., the intestines). The nested systems can be disposable and low-cost. In some embodiments, the outer rigidizing device can be a dynamically rigidizing overtube (e.g., as described in PCT/US18/42946, the entirety of which is incorporated by reference herein). In some embodiments, the inner rigidizing device can be a rigidizing system or a commercially available scope, for example a 5 mm diameter nasal scope. Utilizing rigidization and a nested system enables the utilization of a smaller scope that delivers, compared to a duodenoscope, more flexibility if desired, more stiffness if desired, enhanced maneuverability, and the ability to articulate at a much smaller radius of curvature.

In some embodiments, upon reaching the target destination, the inner rigidizing device of a nested system can be withdrawn. The outer rigidizing device can remain rigidized and contrast can be injected through the inner element's space to fluoroscopically image.

RF coils can be used in any of the nested systems described herein to provide a 3-D representation of whatever shape the nested system takes. That representation can be used to re create a shape or return to a given point (e.g., for reexamination by the doctor after an automated colonoscopy).

In some embodiments, the nested systems described herein can be useful as a complete endoscope, with the internal structure carrying the payload of working channels, pressurization lines, vacuum lines, tip wash, and electronics for lighting and imaging (vision systems, ultrasound, x-ray, MRI).

The nested systems described herein can be used, for example, for colonoscopy. Such a colonoscopy nested system can reduce or eliminate looping. It could eliminate the need for endoscopic reduction. Without looping, the procedure can combine the speed and low cost of a sigmoidoscopy with the efficacy of a colonoscopy. Additionally, colonoscopy nested systems can eliminate conscious sedation and its associated costs, time, risks, and facility requirements. Further, procedural skill can be markedly reduced for such colonoscopy procedures by using the nested systems described herein. Further, in some embodiments, the nested systems described herein can provide automated colonoscopy, wherein a vision system automatically drives the nested system down the center of the colon while looking for polyps. Such an automated system would advantageously not require sedation nor a doctor for the basic exam while allowing the doctor to follow up for further examination if required.

In some embodiments, a rigidizing device as described herein can be configured as a rigidizing guiderail (i.e., a rigidizing obturator or dilator). The rigidizing guiderail can have a guidewire lumen to enable passage over a guidewire and can enable passage of a catheter, such as a large bore catheter, thereover. The rigidizing guiderail in the flexible configuration can be more flexible than the guidewire, enabling reliable trackability with only minimal guidewire disturbance. Once it has reached its target location, the rigidizing guiderail can be rigidized (to varying levels of stiffness, such as a stiffness of 50× or greater than in the flexible configuration), and a large bore catheter can be slid over its outer diameter. Additionally, the rigidizing guiderail can enable the passage of a device through the guidewire lumen. The rigidizing guiderail can also be steerable, which can advantageously enable the rigidizing guiderail to be used to vector the large bore catheter. The rigidizing guiderail can vector the large bore catheter by either rigidizing in a steered direction (to enable passage of the large bore catheter thereover) or by steering with the large bore catheter riding thereover. The surfaces of the inside of the large bore catheter and the inside and outside of the rigidizing guiderail can be specifically designed so as to easily slide relative to each other and relative to the guidewire. For example, the surfaces can include low friction layers (e.g., low friction plastic and elastomers) and/or coatings (e.g., hydrophilic coatings) thereon.

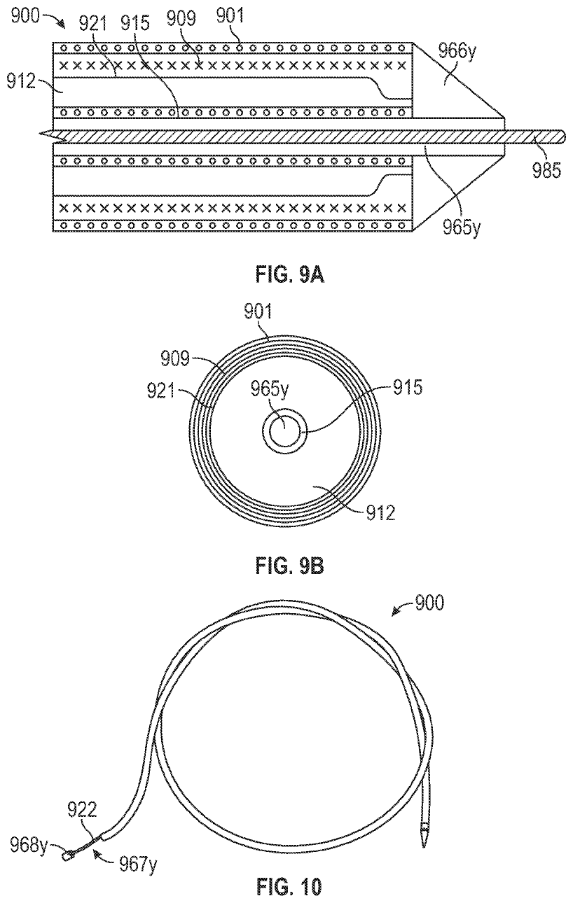

Referring to FIGS. 9A-9B, an exemplary rigidizing guiderail 900 can include an innermost layer 915, a pressure gap 912, a bladder layer 921, a braid layer 909, and an outer outermost layer 901. The layers can terminate in a distal atraumatic tip 966y (e.g., having a taper angle of up to 45 degrees, such as 4-25 degrees, such as 5-15 degrees, such as 5-10 degrees relative to a longitudinal axis of the rigidizing guiderail 900). In some embodiments, the tapered tip 966y can be made of an elastomer. Further, the layers can surround a guidewire lumen 965y configured to allow a guidewire 985 (shown in FIG. 9A) to extend therethrough. The rigidizing guiderail 900 can be configured to rigidize via the application of pressure or vacuum and, correspondingly, to become flexible upon the release of pressure or vacuum as described elsewhere herein.

The wall of the rigidizing guiderail 900 can be relatively thick so as to bridge the gap between the outer diameter of the guidewire 985 and the inner diameter of a large bore catheter placed thereover.

Further, the guidewire 985 can be a standard guidewire, i.e., a guidewire typically used in the associated medical procedure. In some embodiments, the guidewire 985 can have a diameter of 0.01″-0.04″, such as 0.011″, 0.014″, 0.016″, 0.018″, 0.025″, 0.035″, or 0.038″. A large bore catheter can have an inner diameter that is substantially larger, such as 2× or more, 5× or more 8× or more, or 10× or more, than the outer diameter of a guidewire 985. In some embodiments, the large bore catheter can have an inner diameter of 0.02″ to 0.08″, such as 2 FR, 4 FR, 6 FR (0.079″), 8 FR, 10 FR, 12 FR, 14 FR, 16 FR, 20 FR, 24 FR, or 28 FR (0.367″). The rigidizing guiderail 900 can thus advantageously fill the void between the standard guidewire and the large bore catheter.

In some embodiments, the inner diameter of the rigidizing guiderail 900 can be slightly larger than the diameter of the guidewire 985. For example, there can be a radial gap of 0.0005″ to 0.006″ between the inner diameter of the rigidizing guiderail 900 and the diameter of the guidewire 985. Further, the outer diameter of the rigidizing guiderail 900 can be slightly less than the large bore catheter into which it slides. For example, there can be a radial gap of 0.0005″ to 0.015″ between the outer diameter of the rigidizing guiderail 900 and the inner diameter of the large bore catheter.

In some embodiments, the inner diameter of the rigidizing guiderail 900 can be less than 0.05″, such as less than 0.04″, such as less than 0.03″, such as less than 0.02″, while the outer diameter of the rigidizing guiderail 900 can be greater than 0.07″, such as greater than 0.1″, such as greater than 0.15″, such as greater than 0.2″, such as greater than 0.3″. Further, the wall thickness of the rigidizing guiderail 900 can be 0.02″ to 0.2″. The ratio of the inner diameter of the rigidizing guiderail 900 to the outer diameter of the rigidizing guiderail 900 can be less than 50%, such as less than 40%, such as less than 30%, such as less than 25%, such as less than 20%, such as less than 15%, such as less than 10%. Correspondingly, the ratio of the net or double wall thickness to the outer diameter of the rigidizing guiderail 900 can be greater than 50%, such as greater than 60%, such as greater than 70%, such as greater than 75%, such as greater than 80%, such as greater than 85%, such as greater than 90%.

In one exemplary embodiment, a rigidizing guiderail 900 that works with a 0.011″ guidewire and a 6 FR large bore catheter can have a 0.002″ radial clearance, an inner diameter of 0.015″, and an outer diameter of 0.075″ (and thus a 0.030″ wall thickness). In another exemplary embodiment, a rigidizing guiderail 900 that works with both a 0.035″ guidewire and a 28 FR catheter can have 0.002″ radial clearance, an inner diameter of 0.039″ and an outer diameter of 0.363″ (and thus a 0.162″ wall thickness).

Exemplary dimensions (in inches) and percentages are shown below in table 1.

TABLE 1
Sample dimensions of rigidizing guiderail

| | 6 FR | 12 FR | 16 FR | 20 FR | 28 FR |
|---|---|---|---|---|---|
| | O.D. | OD | OD | OD | OD |
| Guidewire diameter | 0.011 | 0.035 | 0.035 | 0.035 | 0.035 |
| Inner diameter | 0.015 | 0.039 | 0.039 | 0.039 | 0.039 |
| Outer diameter | 0.079 | 0.158 | 0.210 | 0.263 | 0.367 |
| Inner diameter/outer | 19% | 25% | 19% | 15% | 11% |
| diameter percentage | | | | | |
| Single wall thickness | 0.032 | 0.059 | 0.085 | 0.112 | 0.164 |
| Net wall thickness | 81% | 75% | 81% | 85% | 89% |
| (double wall)/outer | | | | | |
| diameter | | | | | |

Advantageously, because the inner diameter of the rigidizing guiderail 900 can be relatively small, the innermost layer 915 can be more resistant to collapse (e.g., when pressure is applied) relative to a larger diameter device. As a result, the innermost layer 915 may be a simple extruded tube rather than coil wound (though in some embodiments, the innermost layer 915 may be coil wound).

Further, because the wall of the rigidizing guiderail 900 is relatively thick, the strands of the braid layer 909 can move more easily relative to one another, giving the rigidizing guiderail 900 increased flexibility. As a result, the rigidizing guiderail 900 can have a stiffness (in the unrigidized flexible configuration) that is less than the stiffness of the guidewire 985. For example, the rigidizing guiderail 900 can be ½ as stiff, ⅓ as stiff, ¼ as stiff, or ⅕ as stiff as the guidewire 985 when the rigidizing guiderail 900 is in the flexible configuration. Conversely, the rigidizing guiderail 900 can have a stiffness (in the rigid configuration) that is greater than the stiffness of the guidewire 985. For example, the rigidizing guiderail 900 can be 10× as stiff, such as 20× as stiff, such as 30× as stiff, such as 40× as stiff, such as 50× as stiff, such as 60× as stiff, such as 70× as stiff, such as 80× as stiff as the guidewire 985 when the rigidizing guiderail 900 is in the rigid configuration. For example, the 16 FR rigidizing guiderail 900 in the flexible configuration can have a deflection force of between 4 and 8 grams for a 2″ cantilever length with a 0.25″ deflection while a 0.035″ guidewire 985 can have a deflection force of 8 to 20 grams for a 2″ cantilever length with a 0.25″ deflection. Rigidized, the rigidizing guiderail 900 can have a deflection force of 600 grams. This rigidizing guiderail 900 can, therefore, deliver a rigidization multiple (i.e., between the flexible and rigid configuration) of over 50×, such as over 75×, such as over 100×.

The rigidizing guiderail 900 can slide over a guidewire 985 in a flexible configuration, rigidize (e.g., with the application of pressure or vacuum), and then provide for trackability of a large bore catheter (e.g., a rigidizing catheter, standard catheter, sheath, delivery catheter, or aspiration catheter) thereover. The rigidizing guiderail 900 can thus advantageously be used to navigate a large bore catheter over a guidewire 985 and/or when it is desired to swap multiple devices of differing bore sizes (such as a delivery sheath for an implant or an access sheath for a balloon or guide sheath) without causing movement of the guidewire 985 and/or unacceptable force on the anatomy.

Referring to FIG. 10, in some embodiments, the proximal end of the rigidizing guiderail 900 can include a low-profile hub 967y configured such that the large bore catheter can be passed thereover, i.e., its diameter can be equal to or less than the diameter of the large bore catheter. The hub 967y can include, for example, a valved inflation connector 968y and an inflation line 922. When a pressure source is attached at to the valved inflation connector 968y, pressure or vacuum can travel through the valved inflation connector 968y to the rigidizing guiderail 900 via inflation line 922. The valved inflation connector 968y can include an auto-shut off valve (e.g., a spring-loaded valve or a swabbable valve) so that when the pressure or vacuum source is removed, the wall of the guiderail 900 remains rigidized. To transition the rigidizing guiderail 900 back to the flexible configuration, the pressure or vacuum can be released via the valve. The low-profile huh 967y can further be configured to enable passage of the guidewire 985 therethrough. In some embodiments, the body of the valved inflation connector 968y can further include a lumen therein for passage of the guidewire 985. In other embodiments, the body of the valved inflation connector 968y can have a notch in the side thereof for passage of the guidewire 985. In other embodiments, the guidewire 985 can be configured to pass next to the body of the valved inflation connector 968y. The low-profile hub 967y can advantageously enable connection to the pressure or vacuum source for rigidization of the rigidizing guiderail 900 and/or can enable control of rigidization while still enabling passage of a large bore catheter thereover. In some embodiments, for example, the huh 967y can have a diameter that is the same or smaller than the diameter of the main body of the rigidizing guiderail 900.

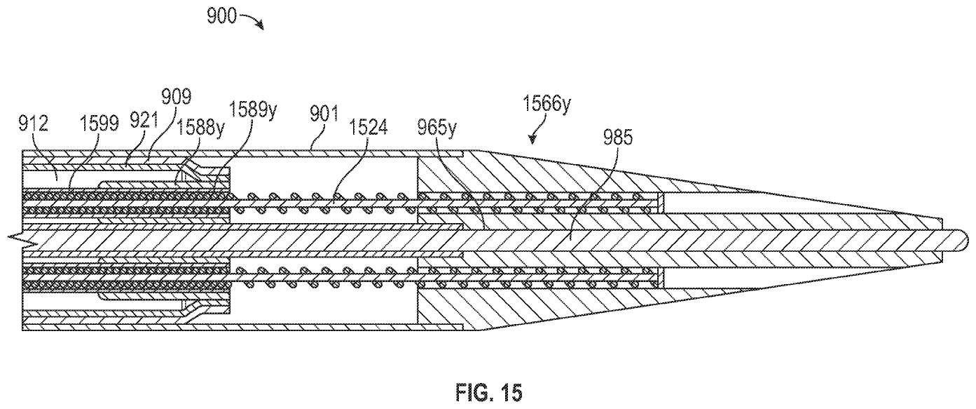

In some embodiments, the wall of the guiderail 900 can include steering elements (e.g., one or more tensile pullwires) to enable steering of the distal end of the guiderail 900. For example, as shown in FIG. 15, a plurality of steering cables 1524 extend through the layered (rigidizing portion) of the guiderail 900 to the distal tip 1566y. The cables 1524 can extend within a jacket 1599, which can be very thin so as to enable simple bending while still protecting the cables 1524 and sealing the cables 1524 from the rigidization pressure or vacuum. Further, coil pipes 1589y extend between the cables 1524 and the jackets 1599, which can advantageously carry the compression loads of the cables 1524 while being of low bending stiffness. The steering cables 1524, when actuated, can cause deflection of the distal tip 1566y towards the side at which the distal end of the cable 1524 is anchored. In some embodiments, as shown in FIG. 15, a bushing 1588y can adhere or otherwise support the ends of the rigidizing layers while allowing the steering cables 1524 to extend therethrough. The coil pipes 1589y can end at the bushing 1588y or can extending further distally into the tip 1566y.

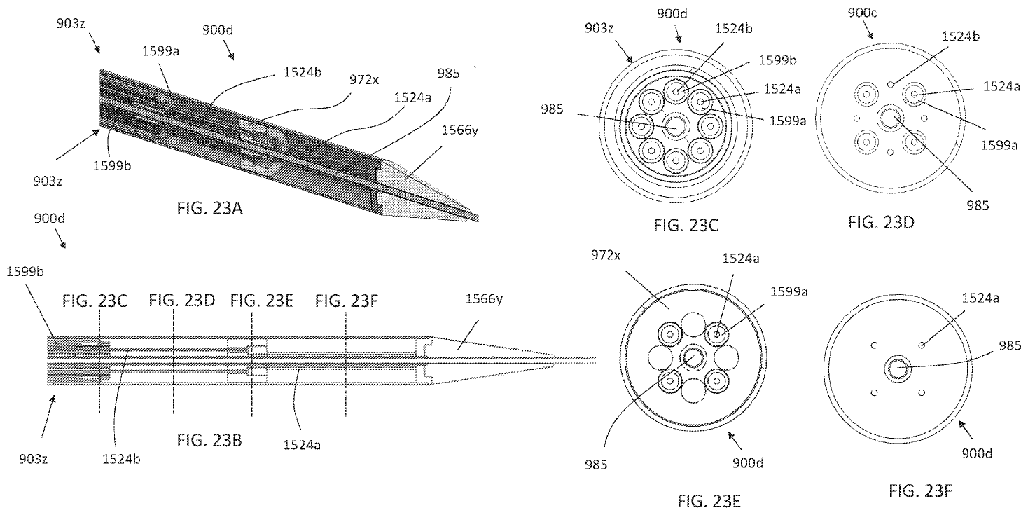

In some embodiments, a rigidizing guiderail 900 can include multiple steering sections. Creating multiple steering sections (for example, one more proximal and one more distal) can enable more complex curves and therefore more sophisticated access and maneuverability. Referring to FIGS. 23A-23F, in some embodiments, a rigidizing guiderail 900d can include a plurality of (e.g., two) steerable sections distal to the main rigidizing elongate body 903z. For example, a set of distal cables 1524a can extend to the distal tip 1566y to provide steerability of the tip 1566y. A set of proximal cables 1524b can extend to a steering collar 972x to provide steerability at the collar 972x (i.e., just proximal to the distal tip 1566y). The steering cables 1524a,b can each have corresponding jackets 1599a,b. Further, the cables 1524a,b can alternate around the circumference (i.e., such that each distal cable 1524a is bordered by two proximal cables 1524b). Advantageously, having a plurality of steering sections can enable the rigidizing guiderail 900d to bend with more degrees of freedom, e.g., to reach particularly tortuous anatomical locations.

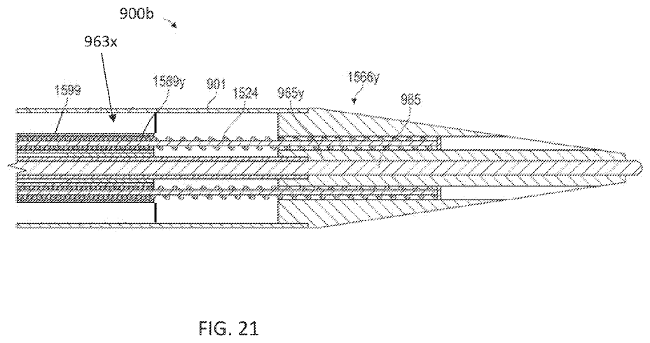

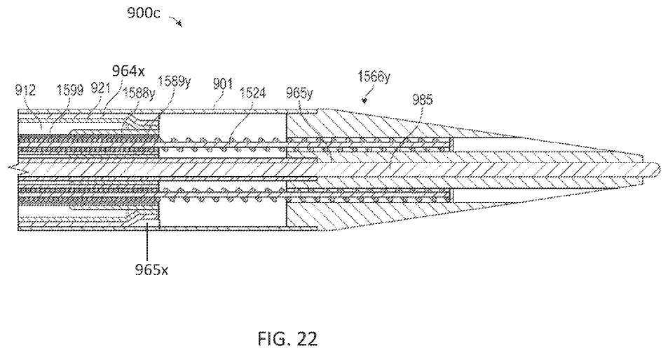

In some embodiments, the rigidizing device 900 can be transitioned from a rigid configuration to a flexible configuration via the application of vacuum or pressure (i.e., rather than transitioning from a flexible configuration to a rigid configuration via the application of vacuum or pressure). In this embodiment, the rigidizing device 900 can therefore be naturally stiff and become flexible through the application of a force (for example, pressure). For example, referring to FIG. 22, a rigidizing device 900c can include a stiffening layer 964x that is biased outwards against layer 901 (i.e., such that the rigidizing device 900c is in a rigid configuration. This outward bias can be created, for example, during manufacturing of the rigidizing device 900c or a portion. For example, the bias can be thermally formed (e.g., for plastics) or biased in the natural condition (e.g., for a metal laser cut design that is nominally crimped smaller for insertion, but that includes material properties that exert an outward force). Upon the application of pressure (e.g., through region 965x), the stiffening layer 964x can be moved away from layer 901, thereby transitioning the device 900c to a flexible state.

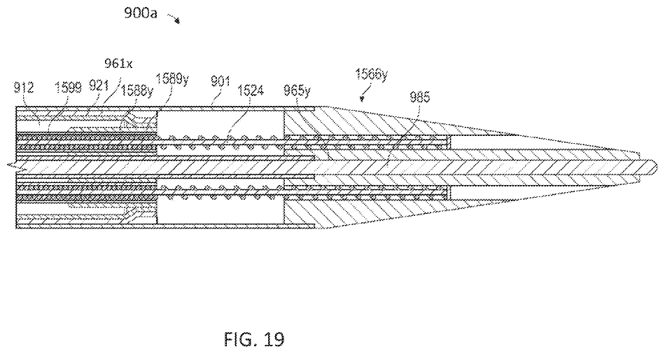

Referring to FIG. 19, in some embodiments (e.g., for rigidizing guiderail 900a), the braid layer 909 can be replaced with a different stiffening layer 961x. The different stiffening layer 961x can include, for example, granules, jamming layers, scales, rigidizing axial members, rigidizers, wedges, laser cut tubes, longitudinal members or substantially longitudinal members that are configured to rigidize via the application of pressure or vacuum.

Referring to FIG. 21, in some embodiments (e.g., for rigidizing guiderail 900b), the braid layer 909, pressure gap 912, and bladder 921 can be replaced with a region 963x configured to be filled with a phase change material such that the rigidizing guiderail 900 can be rigidized via the application of temperature. For example, water in its liquid form can enable a flexible configuration, and water frozen can enable a stiffer configuration. This phase change can also be achieved, for example, by the melting of low temperature plastics or metals. After the heating source is removed, the melted plastics and metals can cool and then solidify, creating a stiffer configuration.

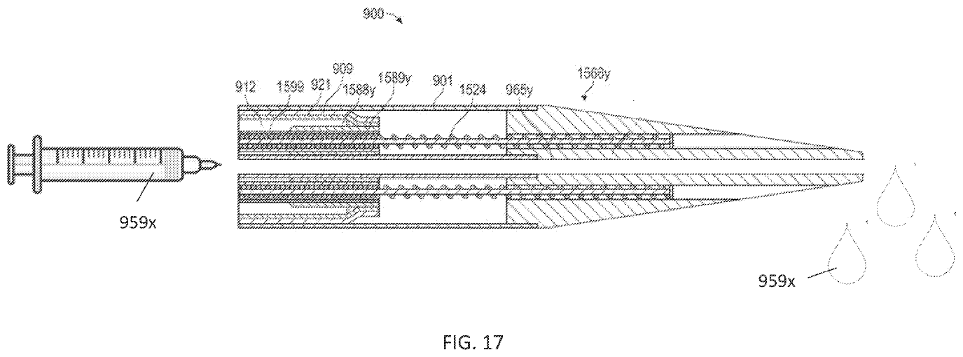

Referring to FIG. 17, in some embodiments, the lumen 965y of the guiderail 900 can be used to puff radiopaque contrast 959x therethrough. Puffing contrast can be used, for example, to image the vasculature with fluoroscopy.

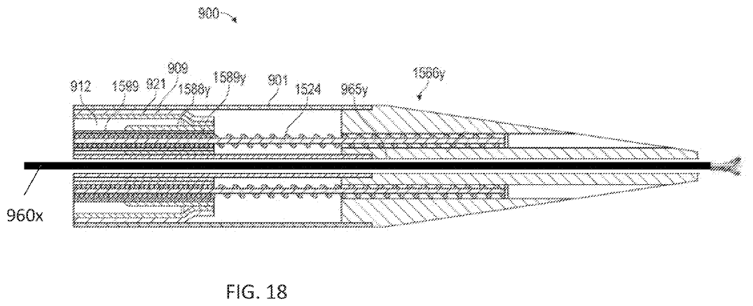

Referring to FIG. 18, in some embodiments, the lumen 965y of the guiderail 900 can be used to pass a biopsy tool 960x, including for myocardial tissue sampling.

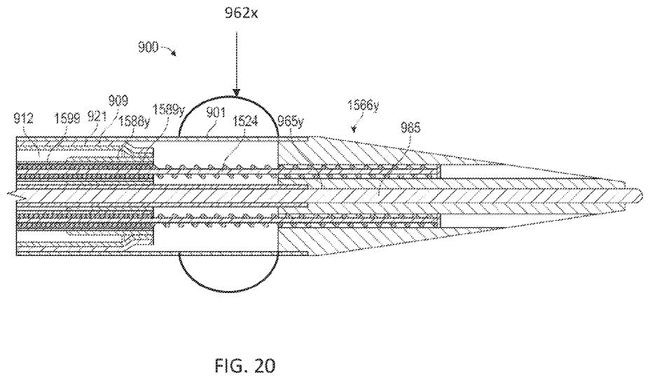

Referring to FIG. 20, in some embodiments, the guiderail 900 can include an expandable balloon 962x thereon or therearound. In some embodiments, the balloon 962x can be at the distal end just proximal to the tapered distal tip 1566y. The unexpanded balloon can be net-flush with the guiderail 900 and/or recessed within the guiderail 900 so as to not impede the passage of catheters over the outer diameter of the guiderail 900. In some embodiments, the balloon 962x can be configured to seal against the vessel, e.g., to grip the vessel. In some embodiments, the balloon 962x can be configured to pass beyond a clot, inflate, and then to pull the clot proximally. In some embodiments, the balloon 962x can be configured to provide distal transport, for example similar to the functionality found in a Swanz-Ganz catheter.

In some embodiments, the guiderail 900 can be part of a robotic system such that its controls (e.g., algorithms and/or actuators) utilize a microprocessor. The guiderail 900 can be used, for example, in conjunction with a rigidizing large bore catheter such that the set creates a nested dual-rigidizing robotic system. The robotic system can drive through the vasculature without the need for direct human contact, thereby providing enhanced control and/or a reduction in radiation exposure (when radiation is used in the procedure) for the user (as the user can be physically distanced from the radiation source).

In some embodiments, the outer diameter of the rigidizing guiderail 900 can include markings or pitch distances thereon such that an imaging system can determine the precise path length through tortuous anatomy (e.g., thereby enabling more optimal selection of a device, such as an AAA graft, to best fit the specific anatomical pathway).

In some embodiments, the guiderail 900 can include calibrated radiopaque or echogenic markers thereon to aid in sizing and/or positioning of the large bore catheters placed thereover.

In some embodiments, the rigidizing guiderail 900 can be preloaded within a large bore catheter (e.g., so that the large bore catheter is not required to fit over the proximal hub).

In some embodiments, the wall of the guiderail 900 can include imaging elements, such as complimentary metal oxide semiconductor (CMOS) sensors, charge-coupled device (CCD) sensors, intracardiac echocardiography (ICE) sensors, or intravascular ultrasound (NUS) sensors therein.

In some embodiments, the rigidizing guiderail 900 can include magnets therein so as to be compatible, for example, with magnetically controlled robotic surgical systems, such as the Sterotaxis Niobe®.

In some embodiments, the lumen of the rigidizing guiderail 900 can be used to place electrodes, such as electrodes for a standard pacemaker. Similarly, the lumen of the guiderail 900 can be used to facilitate placement of a micro-pacemaker (e.g., MDT Micra™) or an electronic sensing device (e.g., CardioMEMS™).

In some embodiments, the wall of the rigidizing guiderail 900 can include one or more sensors (e.g., electromagnetic sensors) therein to track the position of the guiderail 900.

In some embodiments, the stiffness of the rigidizing guiderail 900 can be dynamically modulated (e.g., via modulation of the vacuum or pressure) to assist with advancement and/or pushability. Dynamically modulating the stiffness can advantageously allow adjustment of the stiffness during use (e.g., to increase flexibility or pushability as the rigidizing guiderail 900 is placed through a particularly tortuous locations).

In some embodiments, the rigidizing guiderail 900 can be partially stiffened and/or pre-shaped for specific anatomies or applications, such as for use with a coronary guide sheath. For example, the guiderail 900 can have a built-in directional bias similar to a coronary guide catheter.

Advantageously, the rigidizing guiderail 900 described herein can provide access to anatomy that is deep and tortuous and that cannot be reached with stiffer devices. The rigidizing guiderail 900, with its increased flexibility and relatively thick wall, can enable the delivery of large-bore devices deep in the body. The rigidizing guiderail 900 can also be advantageous where there is poor anatomical support or a lack of a defined anatomical pathway (for example, in the right side of the heart, including passing through the atrium and the ventricle). Finally, the rigidizing guiderail 900 can also be advantageous where high loads on anatomy cause complications (for example, the rigidizing guiderail 900 can prevent pushing on the ventricle, which can otherwise impair cardiac function). The rigidizing guiderails described herein can therefore advantageously enable a wealth of unique procedural advantages and kinematic maneuvers. Moreover, the rigidizing guiderails can be used with a variety of catheters and systems in a broad range of sizes—from micro neurovascular applications up to pulmonary aspiration.

Exemplary use of the rigidizing guiderail 900 (e.g., through the pulmonary vasculature from the right heart into the lungs or in the neurovasculature) is shown in FIGS. 11A-11E. At FIG. 11A, the guidewire 985 is advanced into the vessel 1160z (e.g., towards a clot 1169y). At FIG. 11B, the rigidizing guiderail 900 is advanced over the guidewire 985 in a flexible configuration. The rigidizing guiderail 900 can then be rigidized (e.g., increasing its stiffness at least 1.1 times relative to its initial stiffness) over the guidewire 985. At FIG. 11C, a large bore catheter 970y can be slid over the rigidizing guiderail 900 while the rigidizing guiderail 900 is in the rigid configuration. If the large bore catheter 970y is rigidizing (i.e., includes layers that are rigidized via pressure or vacuum as described elsewhere herein), then the large bore catheter 970y can be rigidized (e.g., increasing its stiffness at least 1.1 times its initial stiffness) after placement over the rigidizing guiderail 900. At FIG. 11D, the rigidizing guiderail 900 can then be transitioned to the flexible configuration and removed from the large bore catheter 970y. The large bore catheter 970y can then be used to perform a procedure (e.g., for aspiration, ablation, withdrawing embolisms, removing tumors, incising, suturing, biopsy, contrast puffing, robotic advancement and control, probing, or interrogating). At FIG. 11E, if desired, an additional catheter 910 (e.g., intermediate-bore device) can be passed through the large bore catheter 970y. The additional catheter 910 (either rigidizing or non-rigidizing) can, for example, pass beyond the distal end of the large bore catheter 970y and be directed towards a bifurcated path (either led by the guidewire or by steering the additional catheter 910). This use of an additional catheter 910 can advantageously enable further penetration of smaller branches with the benefit of a stable base (e.g., a rigidized large bore catheter 970y). In some embodiments, the large bore catheter 970y and the additional catheter 910 or the large bore catheter 970y and the rigidizing guiderail 900 can be alternatively rigidized as shown and described with respect to FIGS. 8A-8H.

In embodiments where the guidewire 985 cannot gain full access on its own (e.g., cannot reach the clot 1169y), the rigidizing device 900 can be used to help the guidewire 985 gain access. For example, a steering portion of the rigidizing device 900 (e.g., as described with respect to FIG. 15) can help to vector or direct the guidewire 985 in the desired direction. This steering can be performed while the rigidizing device 900 is in a fully flexible configuration or while the body of the rigidizing device 900 is rigid and the tip portion is flexible. As another example, the rigidized guiderail 900 can be used to assist with placement of the guidewire 985 by providing support as the guidewire 985 is pushed in the desired direction (e.g., by rigidizing the guiderail 900 at an anatomical branch and pushing the guidewire 985 through the rigidized guiderail 900).

An exemplary method for advancing a guidewire 985 with a steerable rigidizing guiderail 900 can include the following steps:
1. An incision can be made in a vessel, such as in the femoral or iliac vein or internal or external jugular.
2. A guidewire 985 can be introduced through a needle introducer and advanced through the vessel.
3. An introducer sheath with dilator therein can be advanced over the guidewire 985.
4. The dilator can be withdrawn, leaving the introducer sheath in place.
5. A rigidizing guiderail 900 in the flexible configuration can be introduced over the guidewire 985 inside of the introducer.
6. The guidewire 985 can be further advanced (e.g., to a point where it cannot advance any further).
7. The rigidizing guiderail 900 can be advanced over the guidewire 985 and rigidized.
8. The rigidizing guiderail 900 can be steered to point the guidewire 985 in a desired direction.
9. The guidewire 985 can again be advanced (e.g., to a point where it cannot advance any further).
10. The rigidizing guiderail 900 can be made flexible and advanced over the guidewire 985.
11. Steps 6-10 can be repeated until the desired location within the vessel is reached.

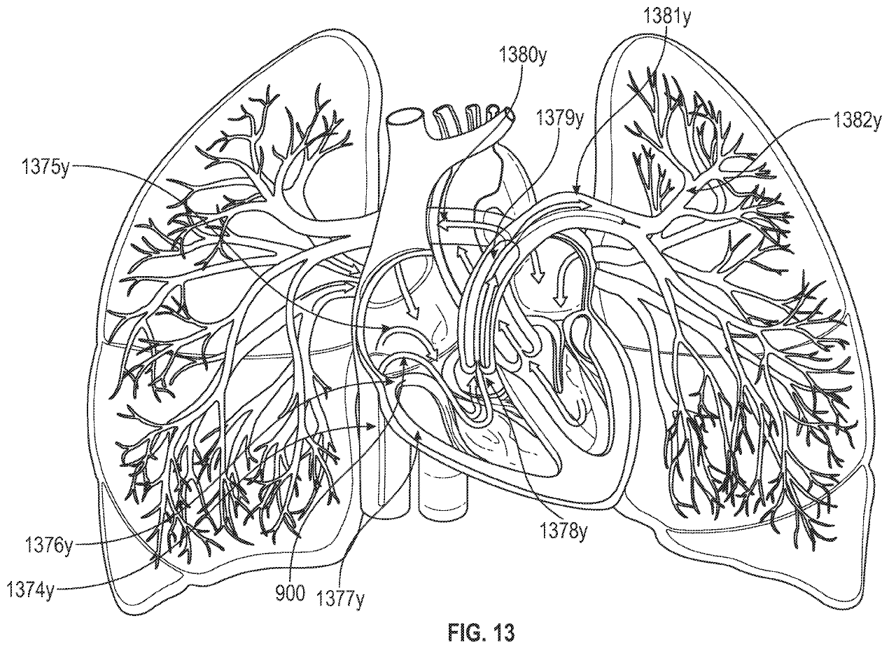

In some embodiments, the rigidizing guiderail 900 can be used for treatment of vascular indications. For example, referring to FIG. 13, in some embodiments, the rigidizing guiderail 900 can be routed through the femoral vein (not shown, but located in an inferior position to the inferior vena cava), to the inferior vena cava 1374y, to the right atrium 1375y, to the tricuspid valve 13′76y, to the right ventricle 1377y, to the pulmonary valve 1378y, to the main pulmonary artery 1379y, to the right pulmonary artery 1380y, to the left pulmonary artery 1381y, and into the deeper branches 1382y of the pulmonary vasculature to perform a procedure in the deep pulmonary vasculature (e.g., for treatment of a pulmonary embolism, or to treat CTEPH (chronic thromboembolic pulmonary hypertension)). In other embodiments, the rigidizing guiderail 900 can be routed through the jugular to the right atrium 1375 and then to the tricuspid valve 1376y, to the right ventricle 1377y, to the pulmonary valve 1378y, to the main pulmonary artery 1379y, to the right pulmonary artery 1380y, to the left pulmonary artery 1381y, and into the deeper branches 1382y of the pulmonary vasculature to perform a procedure in the deep pulmonary vasculature. In other embodiments, the rigidizing guiderail 900 can be used for treatment of aortic disease. The rigidizing guiderail 900 can be used for transcatheter aortic valve replacement (TAVR). The rigidizing guiderail 900 can be used to treat atrial fibrillation. The rigidizing guiderail 900 can be used amidst and within the neurovasculature. The rigidizing guiderail 900 can be used within the pulmonary trunk, amidst and within the pulmonary branches. The rigidizing guiderail 900 can be used in within the chambers of the heart, including the coronary sinus, such as for mitral or tricuspid annuloplasty or placing an arteriovenous shunt.

In some embodiments, the rigidizing guiderail 900 can be used in the gastrointestinal tract, such as the colon. For example, the rigidizing guiderail 900 can be inserted over a guidewire 985 into the colon, a large bore sheath 970y can be placed thereover, and then a colonoscope can be passed through the large bore sheath 970y to enable quick and efficient colonoscopy. As another example, the rigidizing guiderail 900 can be inserted over a string (e.g., put in place via digestion of a pill). As another example, the rigidizing guiderail 900 can be passed over a guidewire 985 with a balloon or net on the distal end thereof (e.g., so that the guidewire 985 is pushed through the colon).

In some embodiments, the rigidizing guiderail 900 can be used in the lungs, for endoscopic procedures (e.g., for endoscopic retrograde cholangiopancreatography), and/or in nasal procedures.

In one specific embodiment, a steerable rigidizing guiderail 900 can be used to treat a pulmonary embolism with aspiration (i.e., a vacuum thrombectomy). An exemplary method for treating the pulmonary embolism with aspiration using a rigidizing large bore catheter 970y and a steerable rigidizing guiderail 900 can include the following steps (with reference to FIGS. 11A-11E and 13):
1. An incision can be made in a vessel, such as in the femoral or iliac vein or internal or external jugular.
2. A guidewire 985 can be introduced through a needle introducer and advanced through the vessel.
3. An introducer sheath with dilator therein can be advanced over the guidewire 985.
4. The dilator can be withdrawn, leaving the introducer sheath in place.
5. The guidewire 985 can be advanced deeper and into the pulmonary arteries 1380y, 1381y. 1382y (methods can include simple wire advancement or floating a Swan-Ganz into the right ventricular outflow tract (RVOT)).
6. A guide catheter can be introduced over the guidewire 985.
7. The guide catheter can be advanced through right atrium 1375y, right ventricle 1377y and through pulmonary valve 1378y into the pulmonary arteries 1380y, 1381y, 1382y.
8. The baseline vitals can be measured and/or contrast can be puffed through the guide catheter to ascertain clot.
9. The guide catheter can be withdrawn.
10. The rigidizing guiderail 900 can be introduced over the guidewire 985 and inside of the introducer.
11. The rigidizing guiderail 900 can be advanced over the guidewire 985 to the target anatomy in the pulmonary arteries (e.g., to the pulmonary embolism).
12. The rigidizing guiderail 900 can be steered to more precisely direct the large bore catheter 970y (subsequently introduced) towards the anatomical target.
13. The rigidizing guiderail 900 can be rigidized.
14. Contrast puff can be provided through the lumen of the rigidizing guiderail 900 to ascertain the clot/embolism. This puffing of contrast can be performed with the guidewire 985 removed or around the guidewire (i.e., within the annular space within the guidewire lumen around the guidewire 985).
15. A rigidizing large bore catheter 970y can be introduced over the guidewire 985, over the rigidizing guiderail 900, and inside of the introducer.
16. The rigidizing large bore catheter 970y can be advanced to target anatomy over the directed rigidizing guiderail 900.
17. The rigidizing large bore catheter 970y can be rigidized.
18. Contrast puff can be provided through the rigidizing guiderail 900 lumen to ascertain the clot.
19. The rigidizing guiderail 900 can be made flexible.
20. The rigidizing guiderail 900 can be withdrawn.
21. An aspiration catheter can be inserted through the rigidizing large bore catheter 970y (alternatively, the large bore catheter 970y can be used directly for aspiration).
22. The aspiration catheter can be precisely navigated through the rigidizing large bore catheter 970y, both axially and rotationally, up to the clot. The aspiration catheter can be advanced with or without the guidewire 985 in place. The aspiration catheter may or may not have obturator. If an obturator was used, it may be removed.
23. The clot can be aspirated into and through aspiration catheter into proximal collection source.
24. Contrast puff can be provided through the aspiration catheter (or rigidizing large bore catheter 970) to examine changes in vitals to determine residual clot burden.
25. If necessary, the aspiration catheter can be moved to a new location and steps 23-24 can be repeated. Obturator may or may not be used in conjunction with aspiration catheter advancement.
26. When the specific vessel, location, or branch is sufficiently treated, the rigidizing large bore catheter 970y can be made flexible.
27. The rigidizing large bore catheter 970y can be moved to a new location as necessary.
28. The rigidizing large bore catheter 970y can be rigidized.
29. Steps 10-28 can be repeated.
30. When sufficient clot has been retrieved or blood loss is at its limit, aspiration can be stopped.
31. The aspiration catheter can be withdrawn.
32. The rigidizing large bore catheter 970y can be made flexible.
33. The rigidizing large bore catheter 970y can be withdrawn.
34. The guidewire 985 (if still present) can be withdrawn.
35. The introducer sheath can be withdrawn.

36. The incision can be closed.

In one embodiment, the relative stiffness of the guidewire 985 can be 8 to 20, the relative stiffness of the rigidizing guiderail 900 can be 4 to 600, the relative stiffness of the rigidizing large bore catheter 970y can be 30-900, and the aspiration catheter can have a fixed stiffness of between 50 and 100 at the proximal end, between 20 and 60 in the middle, and between 10 to 20 at the distal end. If the aspiration catheter were used over the guidewire 985 without the rigidizing guiderail 900 or the rigidizing large bore catheter 970y, the aspiration catheter would cause significant disruption. However, if the rigidizing guiderail 900 is passed over the guidewire 985 while the rigidizing guiderail 900 is in the flexible configuration, it will not disrupt the placement of the guidewire 985. Further, once the guiderail 900 is rigidized, then the large bore catheter 970y in the flexible configuration can be passed over the guiderail 900 without disrupting the shape of the rigidized guiderail 900. Finally, the rigidizing guiderail 900 can be removed and the aspiration catheter passed through the large bore catheter 970y without disruption to the shape, or any displacement within its anatomical location, of the rigidized large bore catheter 970y.

In some embodiments, the method described herein for treating a pulmonary embolism can be modified to include only the use of a rigidizing large bore catheter 970y and aspiration catheter without the rigidizing guiderail 900. An exemplary method for treating a pulmonary embolism with only a rigidizing large bore catheter 970y can include the following steps:
1. An incision can be made in a vessel, such as in the femoral or iliac vein or internal or external jugular.
2. A guidewire 985 can be introduced through a needle introducer and advanced through the vessel.
3. An introducer sheath with dilator therein can be advanced over the guidewire 985.
4. The dilator can be withdrawn, leaving the introducer sheath in place.
5. The guidewire 985 can be advanced deeper and into the pulmonary arteries (methods can include simple wire advancement or floating a Swan-Ganz into the right ventricular outflow tract (RVOT)).
6. A rigidizing large bore catheter 970y with obturator can be advanced over the guidewire 985 and inside of the introducer sheath.
7. The rigidizing large bore catheter 970y with obturator can be advanced through right atrium, right ventricle and through pulmonary valve into the pulmonary artery.
8. Contrast can be puffed through the rigidizing large bore catheter 970y to ascertain the clot.
9. The rigidizing large bore catheter 970y can be advanced to the target anatomy over the guidewire 985.
10. The rigidizing large bore catheter 970y can be rigidized adjacent to the target anatomy.
11. The obturator of the rigidizing large bore catheter 970y can be withdrawn.
12. The guidewire 985 can be removed if needed (because the rigid rigidizing large bore catheter 970y can preserve the location).
13. Contrast can be puffed through rigidizing large bore catheter 970y to ascertain the clot.
14. An aspiration catheter can be introduced through the rigidizing large bore catheter 970y.
15. The aspiration catheter can be precisely navigated final increments, both axially and rotationally, up to the clot.
16. The clot can be aspirated through the aspiration catheter (e.g., into a proximal collection source).
17. The aspiration catheter can be moved to new location and steps 15-16 repeated.
18. Contrast can be puffed through the rigidizing large bore catheter 970y to ascertain the clot.
19. When that branch is sufficiently treated, the rigidizing large bore catheter 970y can be made flexible.
20. The rigidizing large bore catheter 970y in the flexible configuration can be moved.
21. The rigidizing large bore catheter 970y can be rigidized.
22. Steps 15-18 can be repeated.
23. When sufficient clot has been retrieved or blood loss is at its limit, aspiration can be stopped.
24. The aspiration catheter can be withdrawn.
25. The rigidizing large bore catheter 970y can be made flexible.
26. The rigidizing large bore catheter 970y can be withdrawn.
27. The guidewire 985 can be withdrawn.
28. The introducer sheath can be withdrawn.
29. The incision can be closed.

In some embodiments, the rigidizing guiderail 900 and/or rigidizing large bore catheter 970y can include a balloon on a distal end thereof (e.g., as described with respect to FIG. 20). The balloon can be inflated such that blood flow propels the balloon and the rigidizing guiderail 900 or large bore catheter 970y (both in the flexible configuration) through the pulmonary vasculature. For example, the blood flow can propel the balloon (and thus the rigidizing guiderail 900 and/or rigidizing large bore catheter 970y) through the heart and to or through the pulmonary vasculature.

Additionally, and advantageously, use of a rigidizing guiderail 900 and/or a rigidizing large bore catheter 970y as described herein for treatment of a pulmonary embolism with aspiration can enable stability deep in the body, which can enable more precise motion deep in the anatomy. The rigidizing guiderail 900 and/or a rigidizing large bore catheter 970y can enable precise 1:1 motion deep in the body. Advantageously, the rigidizing guiderails 900 and/or rigidizing large bore catheters 970y described herein can be hyper-flexible and include a hyper-lubricious conduit. Further, when advanced to the desired location, the rigidizing guiderail 900 and/or rigidizing large bore catheter 970y can be instantly rigidized, thereby providing a reliable pathway such that the devices can be advanced in more branches, more distally, and with tip locational precision in much closer proximity to the clot for aspiration. This positioning, in turn, can enable a much higher volume of clot for a given volume of blood relative to standard aspiration techniques (i.e., a much higher CBR or Clot to Blood Ratio). The CBR using the techniques described herein can be greater than 0.2, such as greater than 1, such as greater than 2, such as 5 or greater.

In deep right heart procedures, use of a standard guidewire and a standard (non-rigidizing) large-bore catheter can cause hemodynamic compromise by creating regurgitant flow through the tricuspid valve and/or stenotic flow though the pulmonary valve. Hemodynamic compromise can limit the ability of the operation to control the procedure and limits the amount of time available to the operator to perform the throbectomy due to concerns for hemodynamic collapse. The rigidizing guiderails 900 and/or rigidizing large bore catheters 970y as described herein advantageously “lock in” a more favorable anatomical pathway that causes less regurgitant flow and allows the rigidizing guiderails 900 and/or rigidizing large bore catheters 970y to transit the valves in a way reduces hemodynamics strain.

Additionally, the rigidizing guiderails 900 and/or rigidizing large bore catheters 970y described herein can be advantageous for treating a pulmonary embolism for several additional reasons. For example, the rigidizing guiderails 900 and/or rigidizing large bore catheters 970y can be more anatomically conformable and less susceptible to hemodynamic strain, which can enable the operator to stay in the pulmonary arteries longer and have a more controlled procedure. The rigidizing guiderails 900 and/or rigidizing large bore catheters 970y can be positioned in the rigidized configuration directly adjacent to a clot with reduced risk of inadvertent contact with the clot. The ability to remove the guidewire 985 from the rigidizing guiderails 900 and/or rigidizing large bore catheters 970y while performing aspiration can reduce the risk of perforation with the guidewire 985 and improves the cross-sectional area of the aspiration catheter.

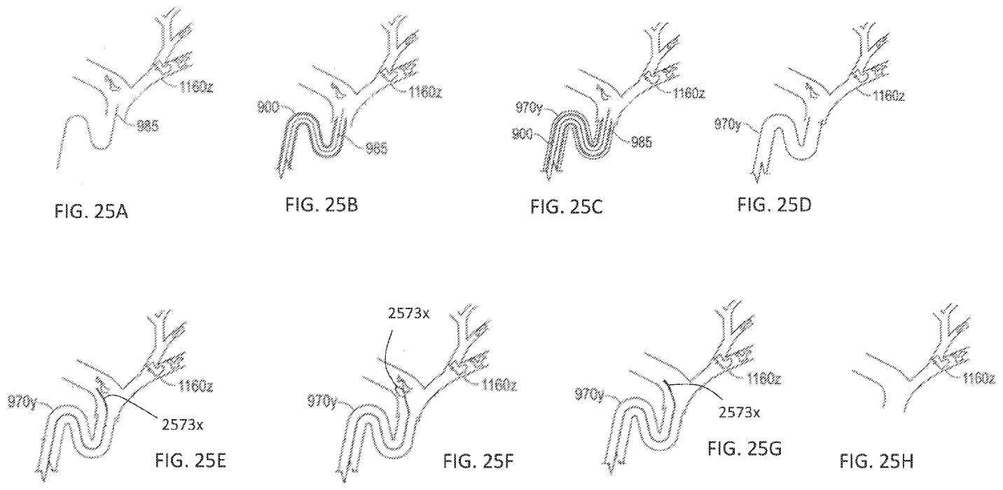

Referring to FIGS. 25A-25H, in another specific embodiment, a rigidizing guiderail 900 can be used to treat chronic thromboembolic pulmonary hypertension (CTEPH). An exemplary method for treating CTEPH using a rigidizing large bore catheter 970y and a steerable rigidizing guiderail 900 can include the following steps:
1. An incision can be made in a vessel 1160z, such as in the femoral or iliac vein or internal or external jugular.
2. A guidewire 985 can be introduced through a needle introducer and advanced through the vessel (FIG. 25A).
3. An introducer sheath with dilator therein can be advanced over the guidewire 985.
4. The dilator can be withdrawn, leaving the introducer sheath in place.
5. The guidewire 985 can be advanced deeper and into the pulmonary arteries (methods can include simple wire advancement or ‘floating’ a Swan-Ganz into the right ventricular outflow tract (RVOT)).
6. A guide catheter can be introduced over the guidewire 985.
7. The guide catheter can be advanced through tight atrium, right ventricle and through pulmonary valve into the pulmonary arteries.
8. The baseline vitals can be measured and then contrast can be puffed to ascertain clot.
9. The guide catheter can be withdrawn.
30. The rigidizing guiderail 900 can be introduced over the guidewire (FIG. 25B) and inside of the introducer.
10. The rigidizing guiderail 900 can be advanced over the guidewire 985 to the target anatomy in the pulmonary arteries (e.g., to the pulmonary embolism or clot).
11. The rigidizing guiderail 900 can be steered to more precisely direct the large bore catheter 970y (subsequently introduced) towards the anatomical target.
12. The rigidizing guiderail 900 can be rigidized.
13. Contrast puff can be provided through the rigidizing guiderail 900 to ascertain the clot/embolism.
14. A rigidizing large bore catheter 970y can be introduced over the guidewire 985 (FIG. 25C), over the rigidizing guiderail 900, and inside of the introducer.
15. The rigidizing large bore catheter 970y can be advanced to target anatomy over the directed rigidizing guiderail 900.
16. The rigidizing large bore catheter 970y can be rigidized.
17. Contrast puff can be provided through the rigidizing guiderail 900 lumen to ascertain clot.
18. The rigidizing guiderail 900 can be made flexible.
19. The rigidizing guiderail 900 and guidewire 985 can be withdrawn (FIG. 25D).
20. A CTEPH balloon 2573x can be introduced through the rigidized large bore catheter 970y (FIG. 25E).
21. The CTEPH balloon 2573x can be advanced and precisely navigated final increments up to the clot or stenosis.
22. The CTEPH balloon insufflator can be prepared.
23. The CTEPH balloon 2573x can be inflated (FIG. 25F).
24. The CTEPH balloon 2573x can be deflated.
25. Contrast can be provided through the large bore catheter 970y, and changes in vitals can be reviewed to determine residual clot burden.
26. The CTEPH balloon 2573x can be moved to new location and steps 22-26 repeated.
27. When the branch or vessel of interest is sufficiently treated, the rigidizing large bore catheter 970y can be made flexible.
28. The rigidizing large bore catheter 970y can be moved to a new location as desired.
29. The rigidizing large bore catheter 970y can be rigidized.
30. Steps 11-29 can be repeated.
31. When improvement has been made (FIG. 25G) or fluoroscopy time is at its limit, inflation of the CTEPH can be stopped.
32. The CTEPH balloon 2573x can be removed.
33. The rigidizing large bore catheter 970y can be made flexible.
34. The rigidizing large bore catheter 970y can be removed from the anatomy (FIG. 25H).
35. The guidewire 985 can be removed.
36. The introducer sheath can be withdrawn.
37. The incision can be closed.

Patients with CTEPH typically have multiple locations throughout the lungs that require intervention, and accessing those different locations using traditional methods can be extremely time consuming and require extremely long exposures to radiation. In contrast, the rigidizing guiderails 900 and/or rigidizing large bore catheters 970y described herein can be advantageous for treating CTEPH for several additional, reasons. For example, the rigidizing guiderails 900 and/or rigidizing large bore catheters 970y can be more anatomically conformable and less susceptible to hemodynamic strain, which can enable the operator to stay in the pulmonary arteries longer and have a more controlled procedure. The rigidizing guiderails 900 and/or rigidizing large bore catheters 970y can be used to precisely access the desired branch of the pulmonary vasculature to clear a blockage.

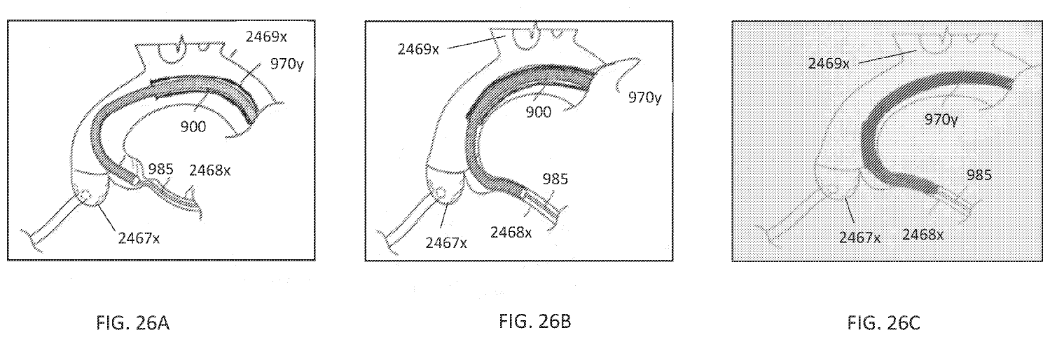

Referring to FIGS. 26A-26C, in another specific embodiment, a steerable rigidizing guiderail 900 can be used for transcatheter aortic valve replacement (TAVR). An exemplary transfemoral method for TAVR using a rigidizing guiderail 900 and a rigidizing large bore catheter 970y can include the following steps:
1. An incision can be made in a vessel, such as in the femoral or iliac artery.
2. A guidewire 985 can be introduced through a needle introducer and advanced through the vessel.
3. An introducer sheath with dilator therein can be advanced over the guidewire 985.
4. The dilator can be withdrawn, leaving the introducer sheath in place.
5. The guidewire 985 can be inserted deeper into the coronary arteries 2468x to the target location at the aortic valve 2467x. In some embodiments, guide catheters and/or specific guidewire shapes can be used to assist in reaching the target location.
6. A pigtail catheter can be inserted over the guidewire 985 or from a separate access site, and contrast can be puffed through pigtail to visualize the anatomy.
7. The guidewire 985 can be inserted fully into the left ventricle with a partial loop in the ventricle.
8. The flexible rigidizing guiderail 900 can be inserted over the guidewire 985 into the ascending aorta.
9. The rigidizing guiderail 900 and guidewire 985 can be pulled to move the rigidizing guiderail 900 off of the aortic arch 2469×.
10. The rigidizing guiderail 900 can be rigidized.
11. A rigidizing large bore catheter 970y in the flexible configuration and with no obturator can be advanced over the rigidizing guiderail 900 (FIG. 26A).
12. When the rigidizing large bore catheter 970y is in the desired location, it can be rigidized (FIG. 26B).
13. The rigidizing guiderail 900 can be made flexible and removed from the body (FIG. 26C).
14. A delivery catheter with a valve implant can be advanced over the guidewire 985 through the rigidizing large bore catheter 970y to the target location at the aortic valve 2467x.
15. The valve implant can be deployed through self expansion or balloon dilation.
16. The delivery system can be removed.
17. The rigidizing large bore catheter 970y can be made flexible and removed.

In some embodiments, the method for TAVR can be modified to use only a rigidizing guiderail 900 and not the rigidizing large bore catheter 970y. An exemplary transfemoral method for TAVR using a rigidizing guiderail 900 can include the following steps:
1. An incision can be made in a vessel, such as in the femoral or iliac artery.
2. A guidewire 985 can be introduced through a needle introducer and advanced through the vessel.
3. An introducer sheath with dilator therein can be advanced over the guidewire 985.
4. The dilator can be withdrawn, leaving the introducer sheath in place.
5. The guidewire 985 can be inserted deeper into the anatomy to the target location. In some embodiments, guide catheters and/or specific guidewire shapes can be used to assist in reaching the target location.
6. A pigtail catheter can be inserted over the guidewire 985 or from a separate access site, and contrast can be puffed through pigtail to visualize anatomy.
7. The guidewire 985 can be inserted fully into left ventricle with a partial loop in the ventricle.
8. The rigidizing guiderail 900 in a flexible configuration can be inserted over the guidewire 985 into the ascending aorta.
9. The rigidizing guiderail 900 and guidewire 985 can be pulled to move the rigidizing guiderail 900 off of the aortic arch.
10. The rigidizing guiderail 900 can be rigidized.
11. A delivery device with a valve implant can be inserted over the rigidizing guiderail 900 to the desired location.
12. The valve implant can be deployed through self expansion or balloon dilation.
13. The delivery system can be removed.
14. The rigidizing guiderail 900 can be made flexible and removed.

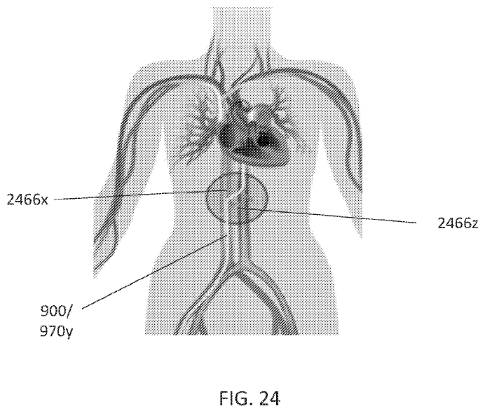

Referring to FIG. 24, an exemplary method for TAVR with transcaval access using a rigidizing guiderail 900 or a rigidizing large bore catheter 970y can include the following steps:
1. An incision can be made in the femoral vein 2466x (or the iliac vein).
2. Additionally, an incision can be made in the femoral or iliac artery.
3. An Outback® or similar catheter can be used to cross from the venous to the arterial circulation (i.e., into the descending aorta 2466x).
4. A guidewire 985 can be introduced on through the femoral or iliac vein (e.g., femoral vein 2466x).
5. A snare can be used on the arterial side to grab a guidewire 985 and pass it into the descending aorta 2466z.
6. An introducer sheath with dilator can be inserted over the guidewire 985 into the femoral vein 2466x (or iliac vein) to expand the opening.
7. An introducer sheath with dilator can be further advanced over the guidewire 985 through the femoral vein 2466x (or iliac vein) and into the descending aorta 2466z.
8. The dilator can be removed.
9. The guidewire 985 can be advanced from the descending aorta 2466z through the aortic valve and into the LV.
10. A rigidizing guiderail 900 or a flexible rigidizing large bore catheter 970y with obturator can be inserted over the guidewire 985 and advanced proximal to the aortic valve in the ascending aorta to protect the aortic arch.
11. The rigidizing guiderail 900 or rigidizing large bore catheter 970y can be rigidized.
12. If the rigidizing large bore catheter 970 is used, the obturator can be removed and a delivery catheter with valve implant can be advanced over the guidewire 985 through the rigidizing large bore catheter 970y to the target location.
13. If the rigidizing guiderail 900 is used, a delivery catheter can be inserted thereover and a valve implant can be inserted therethrough (or preloaded therein).
14. The valve can be deployed from the delivery catheter through self expansion or balloon dilation.
15. The delivery system can be removed.
16. The rigidizing large bore catheter 970y can be made flexible and removed.

In some embodiments, the methods described herein for TAVR can be modified to include only the use of a rigidizing large bore catheter 970y without the dynamically rigidizing guiderail 900.

The use of rigidizing guiderails 900 and/or rigidizing large bore catheters 970y as described herein for TAVR can have several advantages. For example, standard delivery systems and valve implants are relatively stiff compared to the surrounding anatomy. These standard delivery systems and valve implants are difficult to navigate through the aortic arch turn and often end up significantly bumping or scraping the wall. It is not uncommon for the aortic arch to be heavily calcified. Such bumping or scraping can rupture the aortic arch and/or knock those calcifications loose, causing embolic material in the blood. The rigidizing guiderails 900 and/or rigidizing large bore catheters 970y described herein can be extremely flexible and can track over the guidewire 985 without touching the aortic arch. The operator can then pull on the rigidizing guiderails 900 and/or rigidizing large bore catheters 970y to ensure that it is off of the wall. Further, when rigid zed, the rigidizing guiderails 900 and/or rigidizing large bore catheters 970y can allow a relatively stiff delivery catheter or to track over the arch without touching the arch or scraping anything, which in turn can enable a stiff valve implant to track around the arch without touching the arch. The rigidizing guiderails 900 and/or rigidizing large bore catheters 970y can thus provide enhanced control and safety relative to standard TAVR systems. Additionally, the rigidizing guiderails 900 and/or rigidizing large bore catheters 970y can enable use of stiffer valve implants because tracking around the arch is simplified. Additionally, in some embodiments, the rigidizing guiderails 900 and/or rigidizing large bore catheters 970y can provide superior control of the implant delivery catheter, enabling the operator to torque the delivery catheter back and forth with precision to ensure that the valve implant is correctly placed (e.g., to reduce paravalvular leakage and/or minimize changes to flow through the valve or blockage to the coronary arteries). Finally, the rigidizing guiderails 900 and/or rigidizing large bore catheters 970y described herein can also advantageously aid in aligning the device with the annulus by reducing the stiffness of the device in the flexible configuration, enabling a greater range of approach angles.

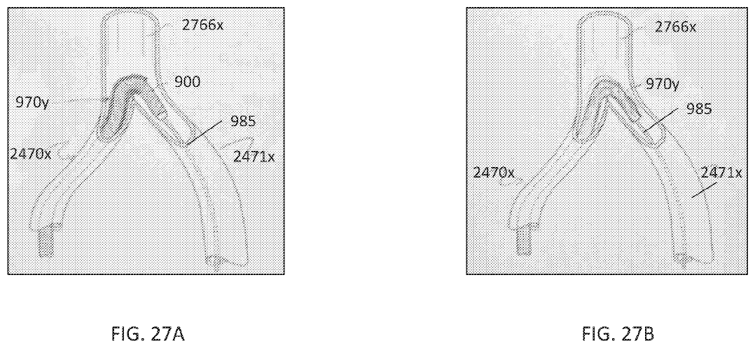

Referring to FIGS. 27A-27B, in another specific embodiment, a steerable rigidizing guiderail 900 can be used to insert an arterial stent up and over the aortic bifurcation. An exemplary method for such placement using a rigidizing guiderail 900 and a rigidizing large bore catheter 970y can include the following steps:
1. An incision can be made in a vessel, such as in the femoral or iliac artery (e.g., the right iliac artery 2470x).
2. A guidewire 985 can be introduced through a needle introducer and advanced through the vessel.
3. An introducer sheath with dilator therein can be advanced over the guidewire 985.
4. The guidewire 985 can be inserted deeper into anatomy to the target location (e.g., in the left iliac artery 2471x). In some embodiments, guide catheters and/or specific guidewire shapes can be used to assist in reaching the target location.
5. The flexible rigidizing guiderail 900 can be inserted over the guidewire 985 and through the introducer sheath (FIG. 27A).
6. The rigidizing guiderail 900 can be advanced to the target location (e.g., proximal to the lesion).
7. The rigidizing guiderail 900 can be rigidized.
8. A rigidizing large bore catheter 970y in the flexible configuration can be advanced over the rigidizing guiderail 900 (FIG. 27B).
9. The rigidizing large bore catheter 970y can be rigidized when the desired location is reached.
10. The rigidizing guiderail 900 can be made flexible and removed from the body.
11. A stent can be inserted through rigidizing large bore catheter 970y and deployed at or near the bifurcation in the descending aorta 2766x.
12. The rigidizing large bore catheter 970y can be made flexible and removed along with the guidewire 985.

In some embodiments, the method of placing an arterial stent up and over and aortic bifurcation can be modified to use only the rigidizing guiderail 900 (and not a rigidizing large bore catheter 970y). An exemplary method for placement of the stent up and over the aortic bifurcation using a rigidizing guiderail 900 can include the following steps:
1. An incision can be made in a vessel, such as in the femoral or iliac artery.
2. A guidewire 985 can be introduced through a needle introducer and advanced through the vessel.
3. An introducer sheath with dilator therein can be advanced over the guidewire 985.
4. The dilator can be withdrawn, leaving the introducer sheath in place.
5. The guidewire 985 can be inserted deeper into the anatomy to the target location (e.g., in another femoral artery). In some embodiments, guide catheters and/or specific guidewire shapes can be used to assist in reaching the target location.
6. A rigidizing guiderail 900 in the flexible configuration can be inserted over the guidewire 985 and through the introducer sheath.
7. The rigidizing guiderail 900 can be advanced to the target location (e.g., proximal to the lesion).
8. The rigidizing guiderail 900 can be rigidized.
9. A delivery sheath can be advanced over the guidewire 985 over the rigidizing guiderail 900.
10. The rigidizing guiderail 900 can be made flexible and removed from body.
11. A stent can be inserted through the delivery sheath and deployed in the desired location.
12. The sheath and guidewire 985 can be removed.

In some embodiments, the method of placing a stent can use a rigidizing guiderail 900 having a balloon thereon (e.g., as described with respect to FIG. 20). An exemplary method for placement of a stent up and over the aortic bifurcation using a rigidizing guiderail 900 having a balloon thereon (and not a rigidizing large bore catheter 970y) can include the following steps:
1. An incision can be made in a vessel, such as in the femoral or iliac artery.
2. A guidewire 985 can be introduced through a needle introducer and advanced through the vessel.
3. An introducer sheath with dilator therein can be advanced over the guidewire 985.
4. The dilator can be withdrawn, leaving the introducer sheath in place.
5. A flexible rigidizing guiderail 900 with deflated balloon thereon can be inserted through the introducer sheath to the femoral artery bifurcation.
6. The balloon can be inflated, and the rigidizing guiderail 900 can be pushed (via blood flow against the balloon) into the adjacent femoral artery.
7. The rigidizing guiderail 900 can be advanced to the target location with the balloon inflated.
8. The balloon can be deflated, and the rigidizing guiderail 900 can be rigidized.
9. A delivery sheath can be advanced over the guidewire 985.
10. The rigidizing guiderail 900 can be made flexible and removed from body.
11. A stent can be inserted through the delivery sheath and deployed.
12. The sheath and guidewire can be removed.

In some embodiments, the above method for placement of a stent up and over the aortic bifurcation using a rigidizing guiderail 900 with a balloon can be modified to additionally include the use of a rigidizing large bore catheter 970y (rather than the delivery sheath).

In some embodiments, the above methods for placement of a stent up and over the aortic bifurcation can be modified to include the use of only a rigidizing large bore catheter 970y rather than a rigidizing guiderail 900.

In the traditional method for placement of a stent up and over the aortic bifurcation, it is often very challenging to get the delivery sheath around the bifurcation from one femoral artery to the adjacent femoral artery. A typical delivery sheath must be stiff enough to carry the stent payload through without deforming and being pushed back into the bifurcation while being flexible enough to get over the bifurcation. As a result, the stiffness of a standard delivery sheath tends to lack performance in one or both areas. In contrast, the rigidizing guiderail 900 and/or rigidizing large bore catheter 970y can advantageously easily track over a guidewire 985 and, once in place, increase its stiffness so that a relatively stiff delivery sheath can track thereover.

In some embodiments, the above methods for placement of a stent up and over the aortic bifurcation can be used for endovascular aneurysm repair (EVAR). For example, the rigidizing guiderail 900 and/or rigidizing large bore catheter 970y can be used to pull a segment of stent grant from the main body of the graft into a contralateral limb with only a single incision.

In another specific embodiment, a rigidizing guiderail 900 can be used for electrophysiology, such as for atrial fibrillation ablation. An exemplary method for electrophysiology using a rigidizing guiderail 900 and a rigidizing large bore catheter 970y can include the following steps:
1. An incision can be made in a vessel, such as in the femoral or iliac artery.
2. A guidewire 985 can be introduced through a needle introducer and advanced through the vessel.
3. An introducer sheath with dilator therein can be advanced over the guidewire 985.
4. The dilator can be withdrawn, leaving the introducer sheath in place.
5. A transeptal needle can be passed over the guidewire 985 and puncture through the septum.
6. The guidewire 985 can then be advanced into the left atrium through the needle.
7. The needle can be removed.
8. The rigidizing guiderail 900 can be advanced over the guidewire and into the left atrium.
9. An access sheath can be advanced over the rigidizing guiderail 900 and positioned in the left atrium.
10. Mapping and/or ablation catheters can be advanced into the left atrium through the rigidizing guiderail 900 or mapping and ablation can be performed through the central lumen of the rigidizing guiderail 900.
11. Upon completion of ablation, the mapping and/or ablation catheters and access sheath and be removed.
12. The rigidizing guiderail 900 can be made flexible and removed.

In some embodiments, the above method of using a rigidizing guiderail 900 for electrophysiology can be modified to use a rigidizing large bore catheter 970y rather than the rigidizing guiderail 900.

The rigidizing guiderail 900 and/or rigidizing large bore catheter 970y for use in electrophysiology can advantageously provide grater accuracy and stability relative to standard techniques. The rigidizing guiderail 900 and/or rigidizing large bore catheter 970y can create a stable channel to pass instrumentation into the left atrium and/or can enable better access sheath placement instead of or in combination with a standard guidewire. Rigidization of the rigidizing guiderail 900 and/or rigidizing large bore catheter 970y can allow higher and more consistent force transmission as the ablation tip contacts the anatomy.

In another specific embodiment, a rigidizing guiderail 900 can be used to remove a clot in the neurovasculature. An exemplary method to remove a clot in the neurovasculature using a rigidizing guiderail 900 and a rigidizing large bore catheter 970y (similar to as shown in FIGS. 11A-11E) can include the following steps:
1. The location of the blockage can be identified by angiography.
2. An incision can be made in a vessel, such as in the femoral artery.
3. A guidewire 985 can be introduced through a needle introducer and advanced through the vessel.
4. An introducer sheath with dilator therein can be advanced over the guidewire 985.
5. The dilator can be withdrawn, leaving the introducer sheath in place.
6. A guidewire 985 can be introduced and advanced part of the way through femoral artery.
7. A rigidizing guiderail 900 can be introduced with a rigidizing large bore catheter 970y preloaded.
8. Contrast injected can be provided through a lumen of the rigidizing guiderail 900 for visualization.
9. The nested rigidizing guiderail 900 and large bore catheter 970y can be alternately advanced and rigidized (as described herein with respect to nested systems) to advance the guiderail 900 and large bore catheter 970y to the clot.
10. Once the tip of the rigidizing guiderail 900 reaches the start of the clot, the rigidizing guiderail 900 can be rigidized and the rigidizing large bore catheter 970y advanced to the blockage and rigidized.
11. The rigidizing guiderail 900 can be transitioned to the flexible state and removed.
12. A mechanical disruptor can be introduced through the rigidizing large bore catheter 970y.
13. Aspiration can be initiated through the rigidizing large bore catheter 970y to suck clot while the mechanical disruptor tool breaks up the clot.

In some embodiments, the above method of treatment in then neurovasculature can be modified to include only the use of a steerable rigidizing guiderail 900 (and not a large bore catheter 970y). An exemplary method for such removal using a steerable rigidizing guiderail 900 can include the following steps:
1. An incision can be made in a vessel, such as in the femoral or iliac artery.
2. A guidewire 985 can be introduced through a needle introducer and advanced through the vessel.
3. An introducer sheath with dilator therein can be advanced over the guidewire 985.
4. The dilator can be withdrawn, leaving the introducer sheath in place.
5. A guidewire 985 can be introduced and advanced part of the way through femoral artery.
6. A steerable rigidizing guiderail 900 preloaded with an aspiration catheter thereover can be introduced over the guidewire 985.
7. Contrast can be injected through a lumen of the rigidizing guiderail 900 for visualization.
8. The distal end of rigidizing guiderail 900 can be articulated and rigidized to advance the guidewire 985 with high accuracy through difficult to navigate turns. Once the guidewire 985 is advanced, the rigidizing guiderail 900 can be unrigidized and advanced (with the aspiration catheter thereover).
9. Once the rigidizing guiderail 900 tip reaches the start of the clot/blockage, the rigidizing guiderail 900 can be rigidized.
10. The aspiration catheter can be advanced up to clot.
11. The rigidizing guiderail 900 can be removed and the guidewire 985 removed.
12. The aspiration catheter can then be activated to remove the clot.

In some embodiments, the aspiration can be performed directly through the rigidizing guiderail 900 or rigidizing large bore catheter 970y rather than inserting a separate aspiration catheter.

Advantageously, the rigidizing guiderail 900 and/or rigidizing large bore catheter 970y can facilitate accessing deeper in the neurovascular anatomy with larger lumen devices. Additionally, use of the steerable rigidizing guiderail 900 can eliminate the need for a sheath and delivery catheter for an aspiration device and/or mechanical disruptor. The rigidized large bore catheter 970y can enable better control of the aspirator and/or mechanical clot disruptors and can provide 1:1 motion deep in the neurovasculature.

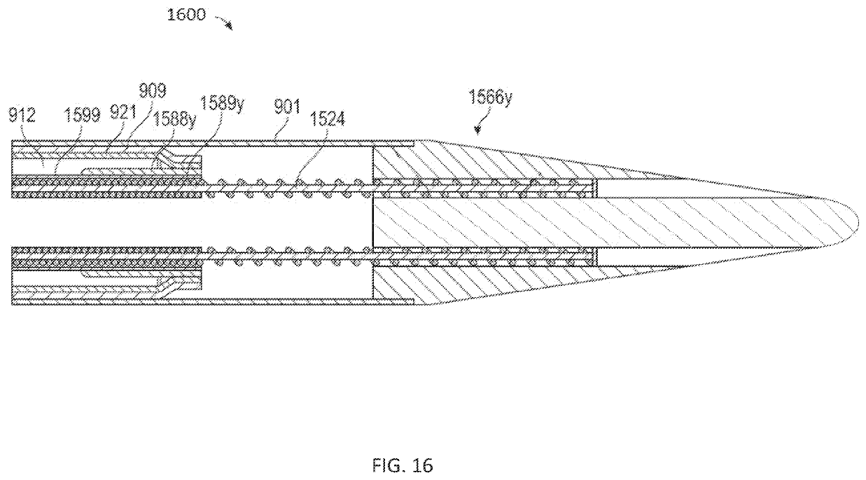

In some embodiments, a rigidizing device as described herein can be configured as a rigidizing guidewire. For example, an exemplary rigidizing guidewire 1600 is shown in FIG. 16. The rigidizing guidewire 1600 can be similar to guiderail 900 except that it can include a solid interior (i.e., without a guidewire lumen). The rigidizing guidewire 1600 can enable passage of a catheter, such as a large bore catheter, thereover. Like the rigidizing guiderail 900, the rigidizing guidewire 1600 can be inserted into the body in a flexible configuration. Once it has reached its target location, the rigidizing guidewire 1600 can be rigidized (e.g., to a stiffness of 50× or greater than in the flexible configuration), and a large bore catheter can be slid over its outer diameter. The rigidizing guidewire 1600 can also be steerable (i.e., via steering cables 1524), which can advantageously enable the rigidizing guidewire 1600 to be used to vector the large bore catheter.

The rigidizing guidewire 1600 can have an outer diameter of 0.01″-0.4″, such as 0.011″, 0.014″, 0.016″, 0.018″, 0.025″, 0.035″, 0.038″, 0.079″, 0.158″, 0.210″, 0.263″, or 0.367″.

The rigidizing guidewire 1600 can be used in place of the guidewire 985 and/or the rigidizing guiderail 900 for any of the methods described herein. Advantageously, because it takes the place of a guidewire in certain procedures, the rigidizing guidewire 1600 can require fewer steps in the procedure (e.g., can eliminate the use of both the guidewire 985 and the guiderail 900). Compared to a standard guidewire, the rigidizing guidewire 1600 offers other important advantages, including variable stiffness (i.e., from hyper-flexible to hyper-stiff) and steerability. Like the rigidizing guiderail 900, the rigidizing guidewire 1600 can be used, for example, for the treatment of vascular indications (e.g., for treatment of a pulmonary embolism, CTEPH, TAVR, atrial fibrillation, aortic disease, the neurovasculature, etc.), in the gastrointestinal tract (e.g., in the colon), or in the lungs.

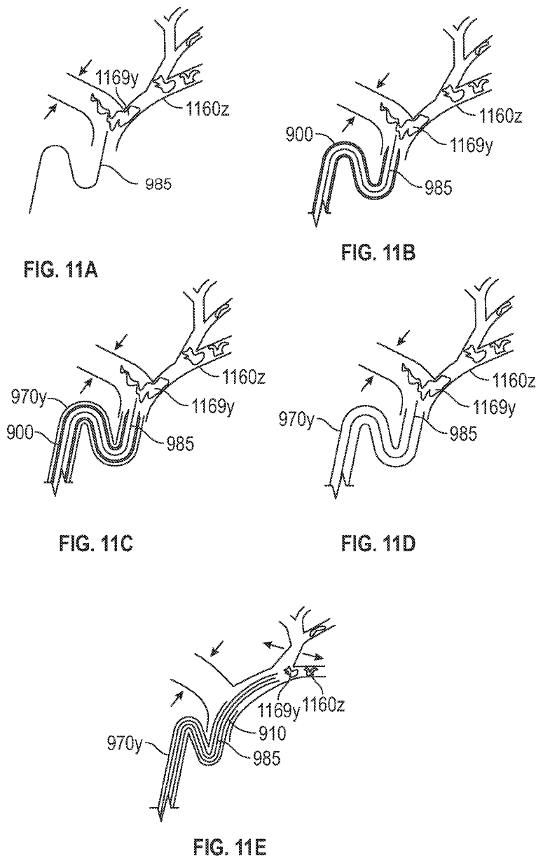

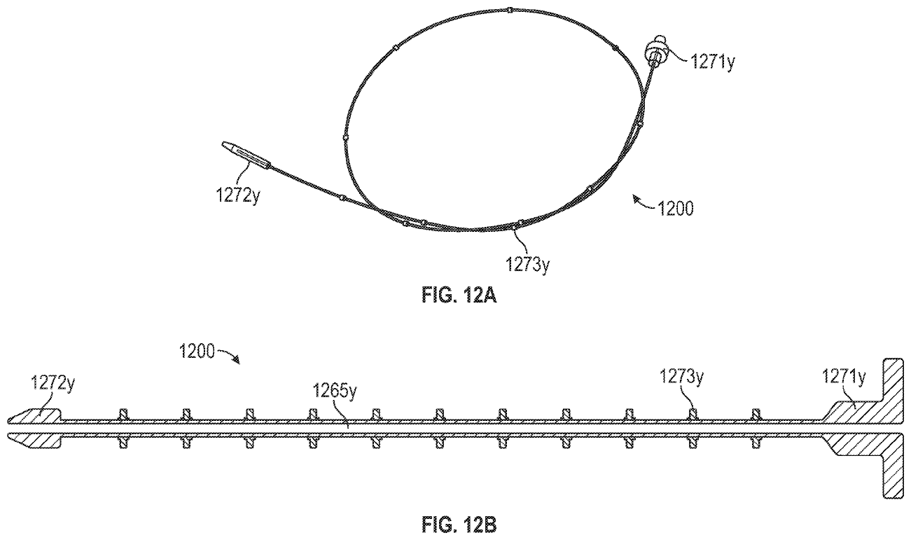

Also described herein are non-rigidizing obturators (or dilators). An exemplary non-rigidizing obturator 1200 is shown in FIGS. 12A-12B. The obturator 1200 is not designed to rigidize, but rather includes a proximal hub 1271y, a guidewire lumen 1265y, and a tapered distal end 1272y. The length of the obturator 1200 can include a plurality of spacers 1273y therealong configured to space the obturator 1200 away from a large bore catheter that is extended thereover. The spacers 1273y can advantageously fill the gap between the guidewire lumen 1265y and a large bore catheter extending thereover (e.g., to prevent buckling of the guidewire lumen 1265y and/or radial movement of the guidewire lumen 1265y within the large bore catheter) without increasing stiffness. The spacers 1273y can be, for example, discs, cones, or spheres. Advantageously, the obturator 1200 has a thin wall that enables high bending flexibility (i.e., minimized bending stiffness) while still allowing a large bore catheter to easily pass thereover.

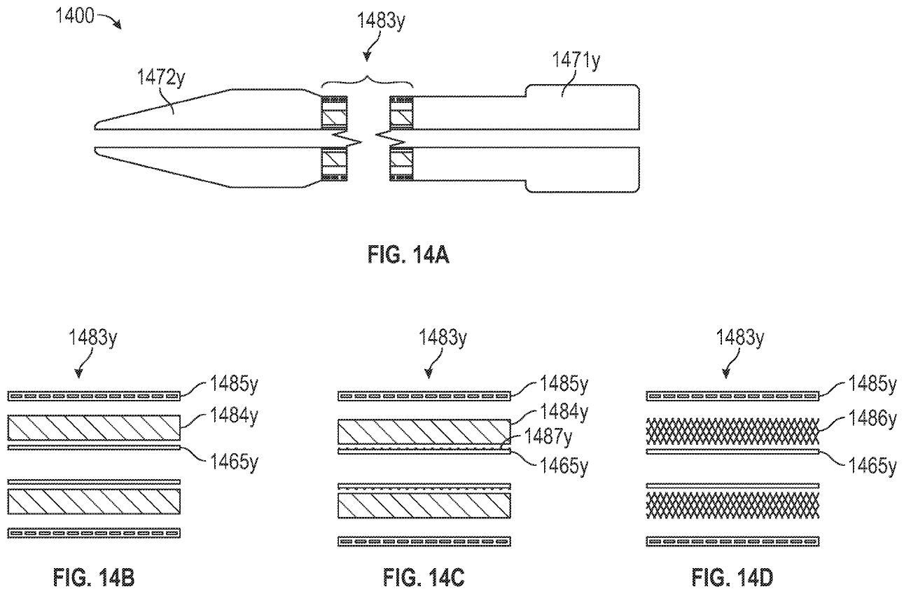

Referring to FIG. 14A, another exemplary non-rigidizing obturator 1400 includes a proximal hub 1471y, a central tube 1483y, and a tapered distal end 1472y. The central tube 1483y includes a plurality of layers positioned around a guidewire lumen 1465y. Referring to FIG. 14A, the central tube 1483y in some embodiments can include an inner low durometer elastomer layer 1484y surrounded by a coil-wound tube layer 1485y. Referring to FIG. 14B, the central tube 1483y in some embodiments can include a spiral tensile element 1487y spiraled around the guidewire lumen 1465y, and then the inner low durometer elastomer layer 1484y and the coil-wound tube layer 1485y. Referring to FIG. 14C, the central tube 1483y in some embodiments can include a braid layer 1486y surrounded by the coil wound tube layer 1485y. The spiral tensile element 1487y can provide tensile stiffness without unduly compromising bend stiffness. The low durometer elastomer layer 1484y can be a low durometer elastomer, such as 50 A or less, such as a silicone (including 30 A or less), latex, or TPE (thermoplastic elastomer). The coil-wound tube layer 1485y can have a tight wire spacing such that it can provide good ‘pushability’ or compression stiffness without unduly compromising the bending stiffness. The combined system (i.e., including the large bore catheter and obturator 1400) can have a bending stiffness contribution from the obturator 1400 that is below 20%, below 15%, below 10%, or below 5% of the combined system stiffness.

It should be understood that any feature described herein with respect to one embodiment can be combined with or substituted for any feature described herein with respect to another embodiment. For example, the various layers and/or features of the rigidizing devices described herein can be combined, substituted, and/or rearranged relative to other layers.

Additional details pertinent to the present invention, including materials and manufacturing techniques, may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are a plurality of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising.” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”. “downwardly”, “vertical”, “horizontal” and the like are used herein for the purpose of explanation only unless specifically indicated otherwise.

Although the terms “fast” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

1.-56. (canceled)

57. A method of performing a medical procedure, comprising:
inserting a guidewire into a body lumen to a desired location;
inserting a rigidizing guiderail over the guidewire while the rigidizing guiderail is in a flexible configuration, wherein the rigidizing guidewire comprises a stiffening layer and a bladder layer,
activating pressure or vacuum when the rigidizing guiderail is proximate to the desired location to transition the rigidizing guiderail to a rigid configuration by driving the bladder layer against the stiffening layer,
removing the guidewire from the central lumen; and performing a medical procedure through the central lumen.

58. The method of claim 57, wherein performing a medical procedure comprises inserting a biopsy tool through the central lumen to gather a tissue sample for biopsy.

59. The method of claim 57, wherein performing a medical procedure comprises aspirating a clot through the central lumen.

60. The method of claim 57, wherein the rigidizing guiderail in the rigid configuration has a higher stiffness than the guidewire, and wherein the rigidizing guiderail in the flexible configuration has a lower stiffness than the guidewire.

61. The method of claim 57, further comprising steering the rigidizing guiderail with a steering element while the rigidizing guiderail is positioned over the guidewire.

62. The method of claim 57, further comprising releasing the pressure or vacuum to transition the rigidizing guiderail back to the flexible configuration.

63. The method of claim 57, wherein the rigidizing guiderail has a radial gap of 0.0005″ to 0.006″ around the guidewire.

64. The method of claim 57, wherein the body lumen is a portion of the pulmonary vasculature.

65. The method of claim 57, wherein the body lumen is a portion of the neurovasculature.

66. The method of claim 57, wherein the body lumen is a myocardium.

67. A method of performing a medical procedure, comprising:
inserting a guidewire into a body lumen to a desired location;
inserting a rigidizing guiderail over the guidewire while the rigidizing guiderail is in a flexible configuration wherein the rigidizing guidewire comprises a stiffening layer and a bladder layer:
when the rigidizing guiderail is proximate to the desired location, activating pressure or vacuum to transition the rigidizing guiderail to a rigid configuration by driving the bladder layer against the stiffening layer,
passing a catheter over the rigidizing guiderail while the rigidizing guiderail is in the rigid configuration; and
performing a medical procedure using the catheter.

68. The method of claim 67, wherein the rigidizing guiderail in the rigid configuration has a higher stiffness than the guidewire, and wherein the rigidizing guiderail in the flexible configuration has a lower stiffness than the guidewire.

69. The method of claim 67, further comprising steering the rigidizing guiderail with a steering element while the rigidizing guiderail is positioned over the guidewire.

70. The method of claim 67, further comprising rigidizing the catheter by activating pressure or vacuum.

71. The method of claim 67, further comprising inserting a third device through the catheter to perform the medical procedure.

72. The method of claim 71, wherein the third device is an aspiration catheter.

73. The method of claim 67, further comprising releasing the pressure or vacuum to transition the rigidizing guiderail back to the flexible configuration.

74. The method of claim 67, further comprising removing the rigidizing guiderail from the catheter prior to performing the medical procedure.

75. The method of claim 67, wherein the rigidizing guiderail has a radial gap of 0.0005″ to 0.006″ around the guidewire.

76. The method of claim 67, wherein the body lumen is a portion of the pulmonary vasculature.

77. The method of claim 76, wherein performing a medical procedure comprises treating for a pulmonary embolism.

78. The method of claim 76, wherein performing a medical procedure comprises treating chronic thromboembolic pulmonary hypertension (CTEPH).

79. The method of claim 67, further comprising puffing contrast through the rigidizing guiderail to identify a clot.

80. The method of claim 67, wherein performing a medical procedure comprises performing transcatheter aortic valve replacement.

81. The method of claim 67, wherein the body lumen comprises an aortic bifurcation.

82. The method of claim 67, wherein performing a medical procedure comprises an electrophysiology procedure.

83. The method of claim 67, wherein the body lumen is a portion of the neurovasculature.

84. A method of treating a pulmonary embolism, comprising:
inserting a guidewire into a body lumen to the pulmonary vasculature proximate to a pulmonary embolism;
inserting a rigidizing guiderail over the guidewire while the rigidizing guiderail is in a flexible configuration, wherein the rigidizing guidewire comprises a stiffening layer and a bladder layer;
transitioning the rigidizing guiderail to a rigid configuration when the rigidizing guiderail is proximate to the pulmonary embolism by driving the bladder layer against the stiffening layer,
passing a catheter over the rigidizing guiderail while the rigidizing guiderail is in the rigid configuration; and
removing at least a portion of the pulmonary embolism through the catheter.
